(19) United States
(12) Patent Application Publication
(10) Pub. No.: US 2005/0084849 A1
(43) Pub. Date: Apr. 21, 2005

(54) Diagnostic polymorphisms for the ecnos promoter

(76) Inventor: David W Moskowitz, St Louis, MO (US)

Correspondence Address: SENNIGER POWERS LEAVITT AND ROEDEL, ONE METROPOLITAN SQUARE 16TH FLOOR, ST LOUIS, MO, 63102, US

(21) Appl. No.: 10/333,878

(22) PCT Filed: Jul. 25, 2001
(86) PCT No.: PCT/US01/23321

Related U.S. Application Data
(60) Provisional application No. 60/220,662, filed on Jul. 25, 2000.

(51) Int. Cl. C12Q 1/68; G06F 19/00; G01N 33/48; H04L 9/32

(52) U.S. Cl. 435/6; 702/20; 713/200

(57) ABSTRACT

Disclosed are single nucleotide polymorphisms (SNIps) associated with breast cancer, lung cancer, prostate cancer, non-insulin dependent diabetes, end stage renal disease due to non-insulin dependent diabetes, hypertension, end stage renal disease F due to hypertension, myocardial infarction, colon cancer, hypertension, atherosclerotic peripheral vascular disease due to hypertension, cerebrovascular accident due to hypertension, cataracts due to hypertension, cardiomyopathy with hypertension, myocardial infarction due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillations without valvular disease, alcohol abuse, anxiety, asthma, chronic obstructive pulmonary disease. cholecystectomy, degenerative joint disease, end stage renal disease and frequent de-clots, end stage renal disease due to focal segmental glomerular sclerosis, end stage renal disease due to insulin dependent diabetes mellitus, or seizure disorder. Also disclosed are methods for using SNPs to determine susceptibility to these diseases; nucleotide sequences containing SNPs; kits for determining the presence of SNPs; and methods of treatment or prophylaxis based on the presence of SNPs.

### BACKGROUND

This invention relates to detection of individuals at risk for pathological conditions based on the presence of single nucleotide polymorphisms (SNPs).

During the course of evolution, spontaneous mutations appear in the genomes of organisms. It has been estimated that variations in genomic DNA sequences are created continuously at a rate of about 100 new single base changes per individual (Kondrashow, J. Theor. Biol. 175:583-594, 1995; Crow, Exp. Clin. Immunogenet., 12:121-128, 1995). These changes, in the progenitor nucleotide sequences, may confer an evolutionary advantage, in which case the frequency of the mutation will likely increase, an evolutionary disadvantage in which case the frequency of the mutation is likely to decrease, or the mutation will be neutral. In certain cases, the mutation may be lethal in which case the mutation is not passed on to the next generation and so is quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphism are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome are single nucleotide polymorphisms or SNPs. SNPs account for approximately 90% of human DNA polymorphism (Collins et al., Genome Res., 8:1229-1231, 1998). SNPs are single base pair positions in genomic DNA at which different sequence alternatives (alleles) exist in a population. In addition, the least frequent allele must occur at a frequency of 1% or greater. Several definitions of SNPs exist in the literature (Brooks, Gene, 234:177-186, 1999). As used herein, the term “single nucleotide polymorphism” or “SNP” includes all single base variants and so includes nucleotide insertions and deletions in addition to single nucleotide substitutions(e.g. A→G). Nucleotide substitutions are of two types. A transition is the replacement of one purine by another purine or one pyrimidine by another pyrimidine. A transversion is the replacement of a purine for a pyrimidine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human. Genet., 60:772-789, 1997; Taillon-Miller et al., Genome Res., 8:748-754, 1998). The frequency of SNPs varies with the type and location of the change. In base substitutions, two-thirds of the substitutions involve the C⇄T (G⇄A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769-776, 1998. SNPs can be detected by restriction fragment length polymorphism (RFLP) (U.S. Pat. Nos. 5,324,631, 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases have also been used to detect SNPs.

There is growing recognition that SNPs can provide a powerful tool for the detection of individuals whose genetic make-up increases their susceptibility to certain diseases. There are four primary reasons why SNPs are especially suited for the identification of genotypes which predispose an individual to develop a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers but as candidates for gene therapy treatments to cure or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

Nitric Oxide (NO) has been recognized as a potential factor in the progression of chronic renal failure (Aiello et al., Kidneys Intl. Suppl., 65:S63-S67, 1998). Nitric oxide, a readily diffusible gas identical to endothelium-derived relaxing factor (EDRF), is synthesized by nitric oxide synthase (NOS). Three isoforms of NOS exist: inducible NOS (iNOS; NOS1), neuronal NOS (nNOS; NOS2), and endothelial constitutive NOS (ecNOS, NOS3).

Nitric oxide, which is vasodilatory, antagonizes the vasoconstrictive effects of angiotensin II and endothelins. Since angiotensin II promotes renal injury, nitric oxide may protect against renal injury from systemic disease such as hypertension or non-insulin dependent diabetes mellitus (NIDDM) (Bataineh et al., Kidney Intl. Suppl., 68:S14-S19, 1998). Nitric oxide has been implicated in the progression of renal disease in rat (Brooks et al., Pharmacology, 56:257-261, 1998) and human (Noris et al., Contrib. Nephrol., 119:8-15, 1996; Kone BC, Am. J. Kidney Dis., 30: 311-333, 1997; Aiello et al., Kidney Int. Suppl., 65:S63-S67, 1998; Raij L., Hypertension, 31:189-193, 1998). The nitric oxide synthase genes are recognized candidate genes for hypertension, renal failure, and cardiovascular disease in general (Soubrier F., Hypertension, 33:924-926, 1999).

L-arginine, a substrate for nitric oxide production, is an essential amino acid that can be given orally. Two studies in rats with subtotal nephrectomy (Reyes et al., Am. J Kidney Dis., 20:168-176, 1992; Ashab et al., Kidney Intl., 47:1515-1521, 1995) have shown improvement of renal function with oral administration of L-arginine, suggesting that low levels of NO may play a role in the development of ESRD. Concentrations of 1.25 to 10 grams/liter of L-arginine were used in the rat studies resulting in a dose of approximately 1.25 to 10 grams/kg body weight/day. In a recent human trial, however, administration of only 0.2 gram/kg body weight/day of L-arginine had no demonstrable effect (De Nicola et al., Kidney Intl., 56:674-684, 1999).

In the remnant kidney model of chronic renal failure in rats, activity of ecNOS remains unchanged whereas the activity of iNOS decreases markedly (Aiello et al., Kidney Intl. 52:171-181, 1997). A deficiency of nitric oxide, especially due to the ecNOS isoform which normally remains unchanged after renal injury, may predispose patients with underlying systemic disease to end-stage renal disease (ESRD) (Huang, Am. J. Cardiol., 82:57S-59S, 1998).

A number of polymorphisms have been reported in the sequence of the ecNOS gene, some of which have also been reported to be associated with variation in plasma levels of NO (Wang et al., Arterioscler. Thromb. Vasc. Biol., 17:3147-3153, 1997; Tsukada et al., Biochem. Biophlys. Res. Commun., 245:190-193, 1998)

Nakayama et al. (Hum. Hered., 45:301-302, 1995; Clin. Genet., 51:26-30, 1997), have reported the presence of highly polymorphic (CA)n repeats in intron 13 of the ecNOS promoter. Bonnardeaux et al. (Circulation, 91:96-102, 1995), reported the presence of two biallelic markers in intron 18 that were not linked to essential hypertension.

Two forms of a 27 base pair repeat in intron 4 have been reported; a larger allele, with 5 tandem repeats, and a smaller allele, with only 4 repeats (third repeat missing). The rare, smaller allele has been associated with coronary artery disease in smokers, but not in patients who had never smoked (Wang et al., Nat. Med., 2:41-45, 1996; Ichihara et al., Am. J. Cardiol., 81:83-86, 199S). The smaller allele has also been associated with essential hypertension (Uwabo et al., Am. J. Hypertens., 11:125-128, 1998). An additional association was also observed in Turkish patients with deep vein thrombosis and strokes (Akar et al., Thromb. Res., 94:63064, 1999). Several studies, however, failed to confirm any association of the intron 4 polymorphism with cardiovascular disease (Yahashi et al., Blood Coagul. Fibrinolysis, 9:405-409, 1998), essential hypertension (Bonnardeaux et al., Circulation, 91:96-102, 1995), or of the ecNOS gene with myocardial infarction (Poirier et al., Eur. J. Clin. Invest., 29:284-290, 1999)

A missense Glutamate 298 to Aspartate variant (E298D) in exon 7 has been associated with coronary spasm in Japanese patients (Yoshimura et al., Hum. Genet., 103:65-69, 1998) as well as enhanced vasoconstriction by phenylephrine (Philip et al., Circulation, 99:3096-3098, 1999). Despite observed associations with coronary spasm (Yoshimura et al., Hum. Genet., 103:65-69, 1998) and preeclampsia, there was no linkage of ecNOS with migraine headaches, which are also thought to involve arterial spasm (Griffiths et al., Neurology, 49:614-617, 1997). The E298D polymorphism was also associated with essential hypertension in some studies (Miyamoto et al., Hypertension, 32:3-8, 1998; Yasujima et al., Rinsho Byori, 46:1199-1204, 1998) but no association was seen in a larger study (Kato et al., Hypertension, 33:933-936, 1999), nor was the E298D polymorphism associated with a measure of aortic stiffiess, a consequence of hypertension (Lacolley et al., J. Hypertens., 16:31-35, 1998). The findings regarding a possible association between the E298D polymorphism and myocardial infarction have been mixed, with an association found in some studies (Hibi et al., Hypertension, 32:521-526, 1998; Shimasaki et al., J. Am. Coll. Cardiol., 31:1506-1510, 1998; Hingorani et al., Circulation, :100:1515-1520, 1999), but not others (Cai et al., J. Mol. Med. 77:511-514, 1999; Liyou et al., Clin. Genet., 54:528-529, 1998). Nor has the E298D polymorphism been associated with cerebrovascular disease in Caucasians (Markus et al., Stroke, 29:1908-1911, 1998; MacLeod et al., Neurology, 53:418-420, 1999).

In view of the contradictory evidence for association with cardiovascular disease of any of the above polymorphisms in ecNOS, the need to focus on functional polymorphisms is clear (Soma, et al., Curr. Opin. Nephrol. Hypertens., 8:83-87, 1999). We therefore searched for functional polymorphisms in the promoter of ecNOS, where single base differences (single nucleotide polymorphisms, or SNPs) can have a major effect on the transcriptional rate of a gene (Cooper D N, Ann. Med., 24:427-437, 1992).

The following polymorphisms in the promoter of ecNOS have been previously described, and are not a subject of this invention. A mutation at position −786 of T to C has been reported which was associated with coronary spasm (Nakayama et al., Circulation, 99:2864-2870, 1999). Also seen were an A-to-G mutation at position −922, and a T-to-A mutation at position −1468, which were linked to the T−786→C mutation. However, in a luciferase construct, only the T−786→C mutation resulted in a significant reduction in ecNOS gene promoter activity. Id. Position −786 corresponds to position +2687 in the promoter sequence contained in GenBank as accession number AF032908 (SEQ ID NO: 1). In this application, bases are numbered from the first transcibed base which is +3473 in AF032908. Thus position −786 corresponds to position +2687 in AF032908 (3473−786=2687).

A MspI restriction fragment length polymorphism (RFLP) has been reported in an Australian Caucasian population (Sim et al., Mol. Genet. Metab. 65:562, 1998). The T to C mutation at position −781 (AF032908 position 2692) was not shown to be associated with any human disease nor to be functional when cloned upstream of a luciferase reporter gene in HepG2 cells.

An additional C to T mutation has also been reported at position −690 (Nishio et al., Biochem. Biophys. Res. Commun., 221:163-168, 1996), corresponding to position +2783 in the promoter sequence AF032908 (Tunny et al., Clin. Exp. Pharmacol Physiol., 25:26-29, 1998).

An ideal approach to disease prevention would be the identification of any genes that predispose an individual to certain diseases early enough to be able to counteract this predisposition.

### SUMMARY OF THE INVENTION

The present inventor has discovered novel single nucleotide polymorphisms (SNPs) within the enodthelial constitutive nitric oxide synthase gene and associated regulatory regions. These polymorphisms are associated with the development of breast cancer, lung cancer, prostate cancer, non-insulin dependent diabetes (NIDDM), end stage renal disease due to non-insulin dependent diabetes (ESRD due to NIDDM), hypertension (HTN), end stage renal disease due to hypertension (ESRD due to NIDDM), myocardial infarction (MI) (collectively known herein as the “Group I Diseases”), colon cancer, hypertension (HTN), atherosclerotic peripheral vascular disease due to hypertension (ASPVD due to HTN), cerebrovascular accident due to hypertension (CVA due to HTN), cataracts due to hypertension (cataracts due to HTN), cardiomyopathy with hypertension (HTN CM), myocardial infarction due to hypertension (MI due to HTN), non-insulin dependent diabetes mellitus (NIDDM), atherosclerotic peripheral vascular disease due to non-insulin dependent diabetes mellitus (ASPVD due to NIDDM), cerebrovascular accident due to non-insulin dependent diabetes mellitus (CVA due to NIDDM), ischemic cardiomyopathy (ischemic CM), ischemic cardiomyopathy with non-insulin dependent diabetes mellitus (ischemic CM with NIDDM), myocardial infarction due to non-insulin dependent diabetes mellitus (MI due to NIDDM), atrial fibrillation without valvular disease (afib without valvular disease), alcohol abuse, anxiety, asthma, chronic obstructive pulmonary disease (COPD), cholecystectomy, degenerative joint disease (DJD), end stage renal disease and frequent de-clots (ESRD and frequent de-clots), end stage renal disease due to focal segmental glomerular sclerosis (ESRD due to FSGS), end stage renal disease due to insulin dependent diabetes mellitus (ESRD due to IDDM), or seizure disorder (collectively known herein as the “Group II Diseases”). (To the extent that hypertension and non-insulin dependent diabetes are included in Group II as well as Group I, it is only for purposes of calculating odds ratios for diseases in Group II related to hypertension and non-insulin dependent diabetes). As such, these polymorphisms provide a method for diagnosing a genetic predisposition for the development of these diseases in individuals. Information obtained from the detection of SNPs associated with the development of these diseases is of great value in their treatment and prevention.

Accordingly, one aspect of the present invention provides a method for diagnosing a genetic predisposition for breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder in a subject, comprising obtaining a sample containing at least one polynucleotide from the subject, and analyzing the polynucleotide to detect a genetic polymorphism wherein said genetic polymorplism is associated with an increased risk of developing these diseases.

Another aspect of the present invention provides an isolated nucleic acid sequence comprising at least 10 contiguous nucleotides from SEQ ID NO: 1, or its complement, wherein the sequence contains at least one polymorphic site associated with a disease and in particular breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder.

Yet another aspect of the invention is a kit for the detection of a polymorphism comprising, at a minimum, at least one polynucleotide of at least 10 contiguous nucleotides of SEQ ID NO: 1, or its complement, wherein the polynucleotide contains at least one polymorphic site associated with a disease condition or disorder, and in particular breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder.

Yet another aspect of the invention provides a method for treating a disease, condition or disorder in a subject, comprising obtaining a sample of biological material containing at least one polynucleotide from the subject; analyzing the polynucleotide to detect the presence of at least one polymorphism associated with the disease, condition or disorder; and treating the subject in such a way as to counteract the effect of any such polymorphism detected.

Still another aspect of the invention provides a method for the prophylactic treatment of a subject with a genetic predisposition to a disease, condition or disorder comprising, obtaining a sample of biological material containing at least one polynucleotide from the subject; analyzing the polynucleotide to detect the presence of at least one polymorphism associated with the disease, condition or disorder; and treating the subject in such a way as to counteract the effect of any polymorphism detected.

Further scope of the applicability of the present invention will become apparent from the detailed description and drawings provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### BRIEF DESCRIPTION OF THE DRAWINGS

These and other features, aspects, and advantages of the present invention will become better understood with regard to the following description, appended claims, and accompanying drawings where:

FIG. 1 shows SEQ ID NO: 1, the nucleotide sequence of the ecNOS gene as contained in GenBank (accession no. AF032908). Position of the single nucleotide polymorphism (SNP) is given using GenBank Accession Number AF032908 as the reference sequence. The first transcribed base is at position +3473 according to the numbering scheme of AF032908; the first translated base (the “A” of the ATG codon for Methionine) is at position +3494. Thus, position +637 according to the numbering scheme of AF032908 corresponds to position −2836 using the traditional numbering scheme, where +1 is the start of transcription. To translate from the numbering scheme of sequence AF032908 to the nucleotide's position relative to the transcription start site, simply subtract 3473 from the indicated position number, i.e. +637 (according to AF032908)−3473=−2836 (according to transcription start site).

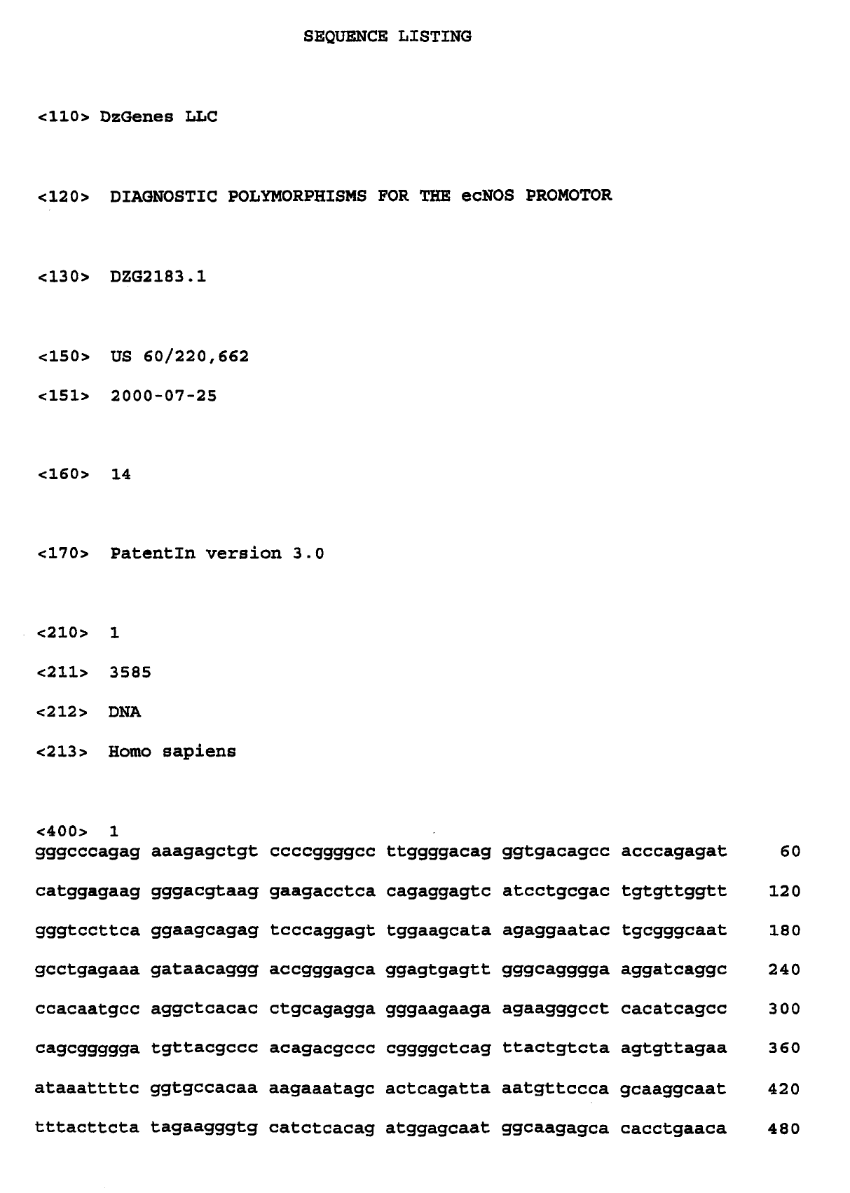

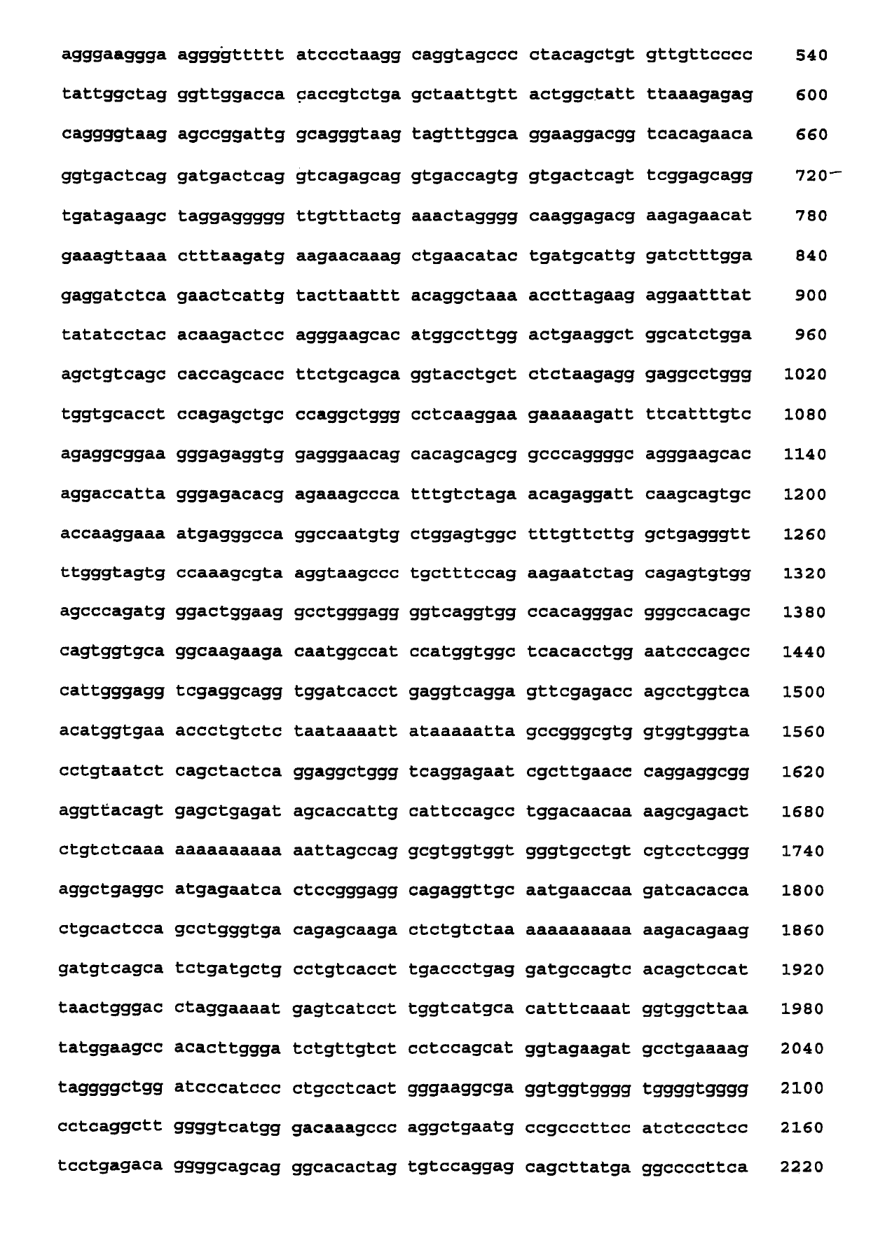

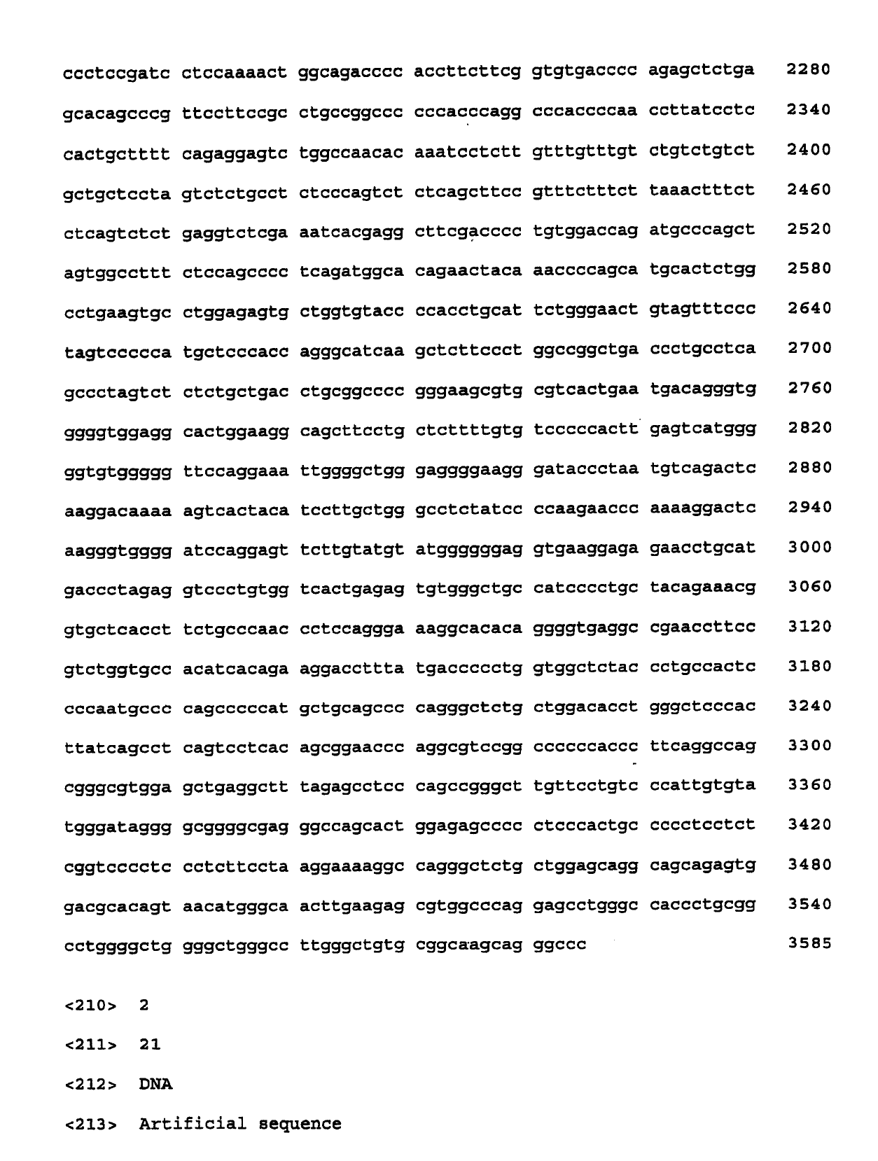

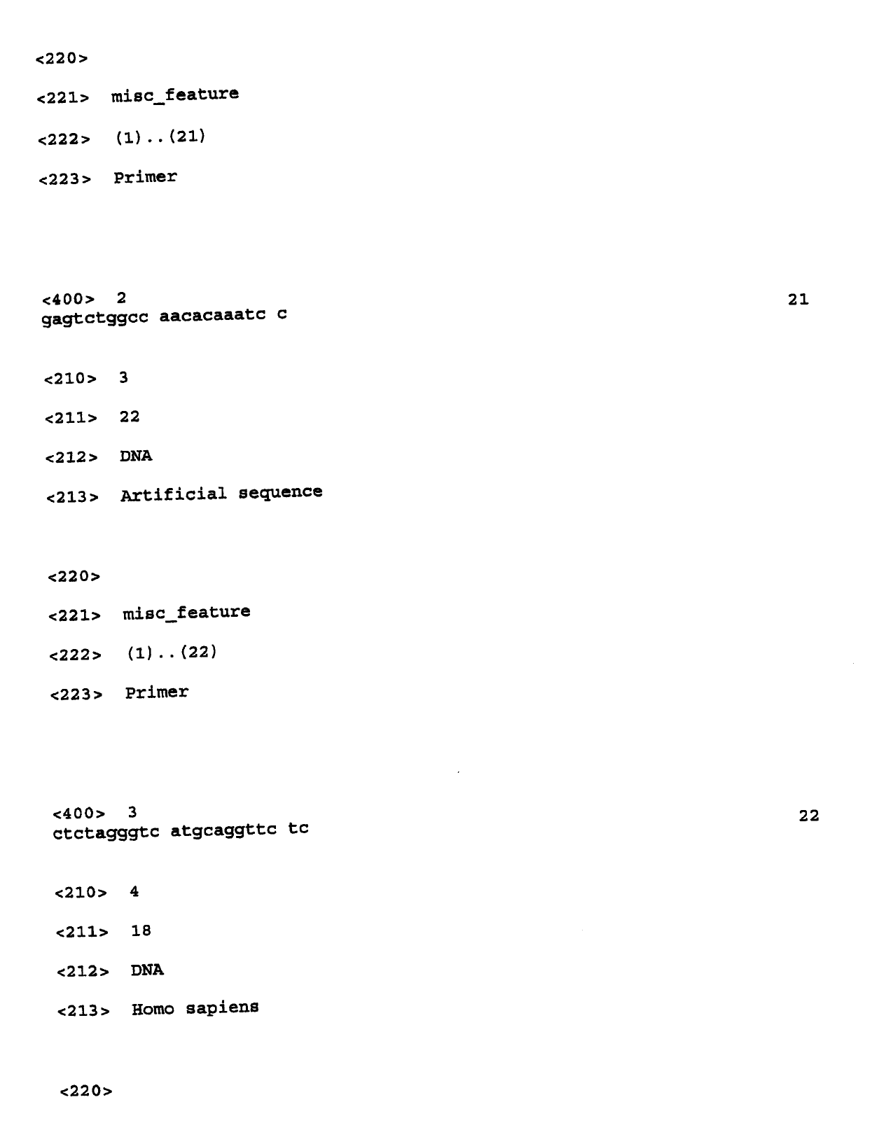

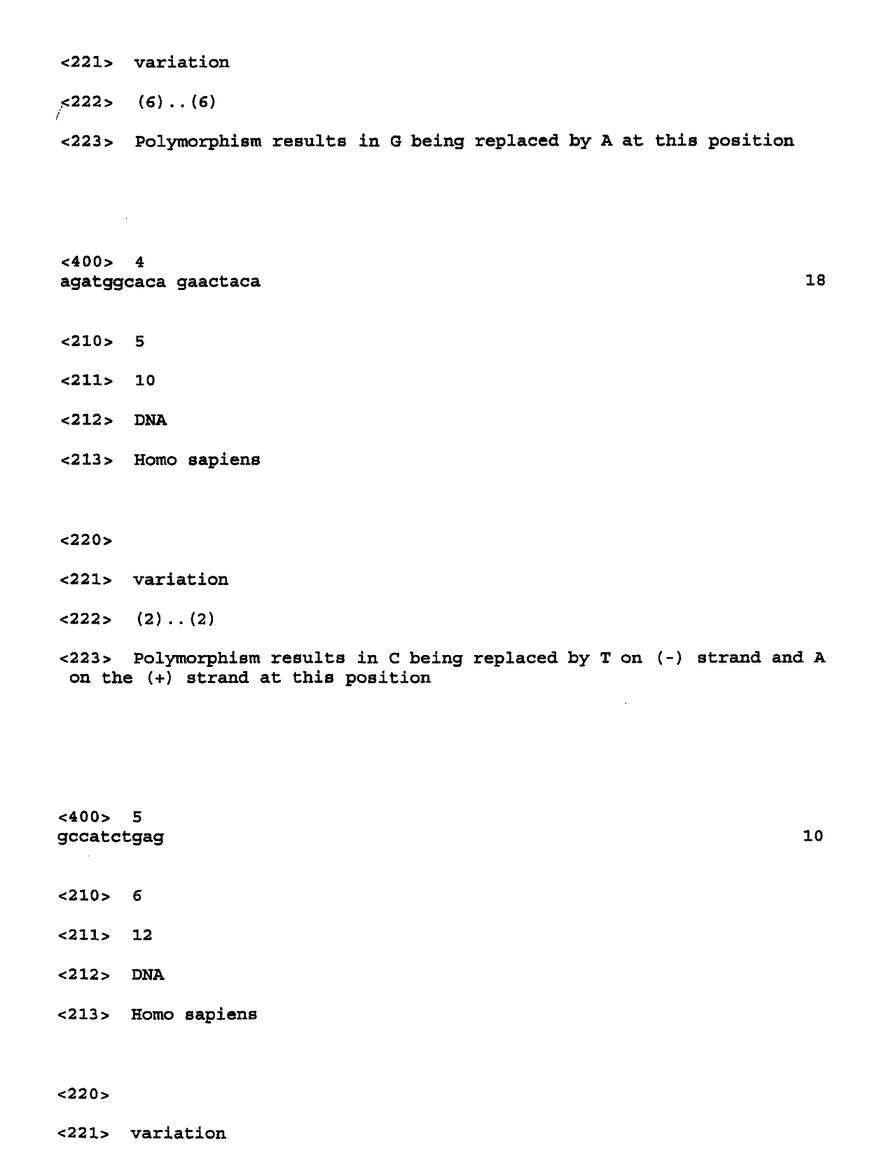

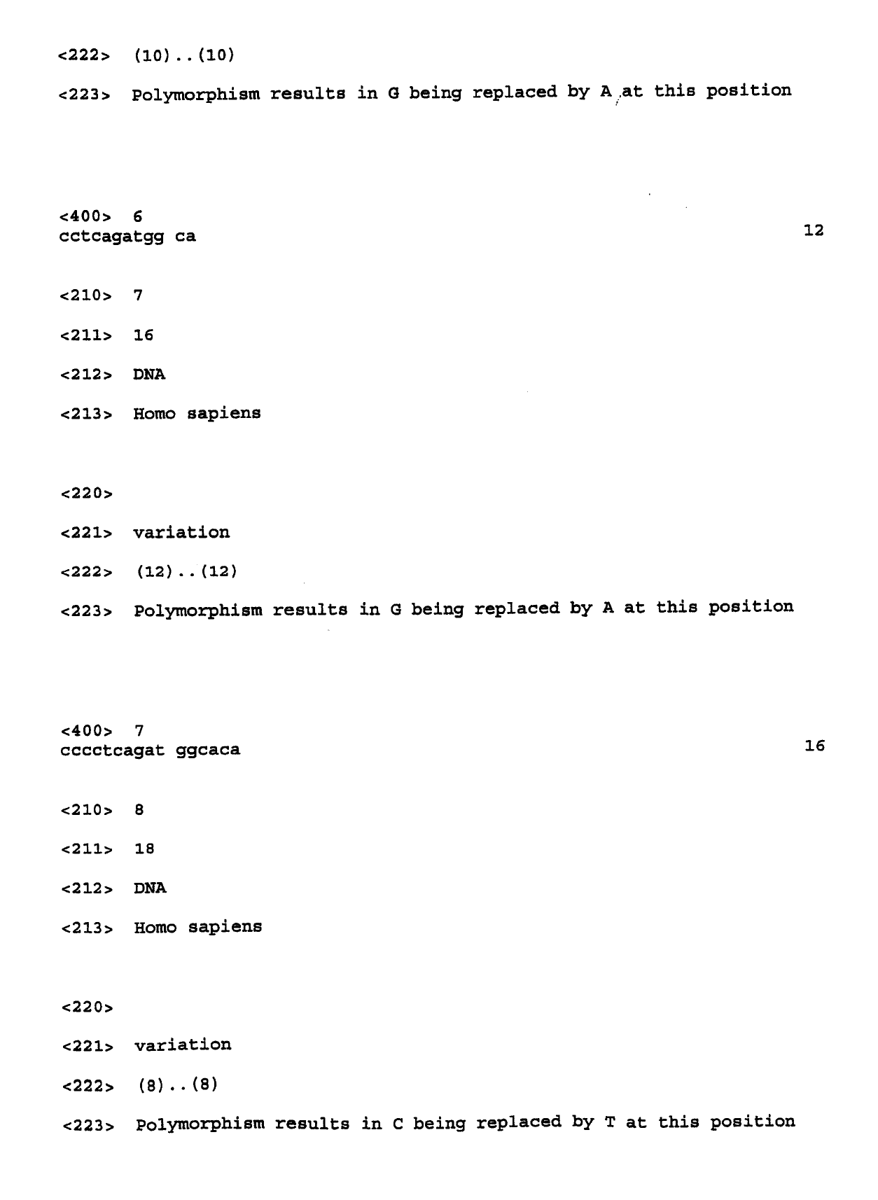

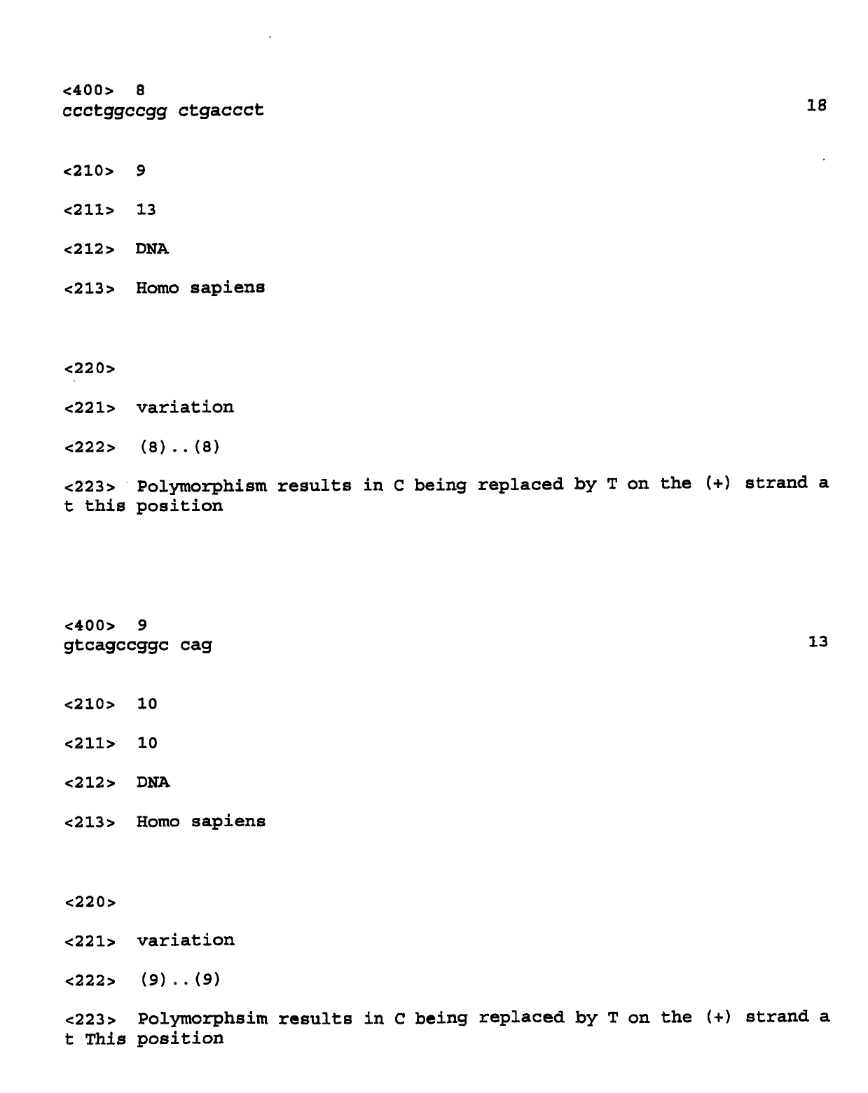

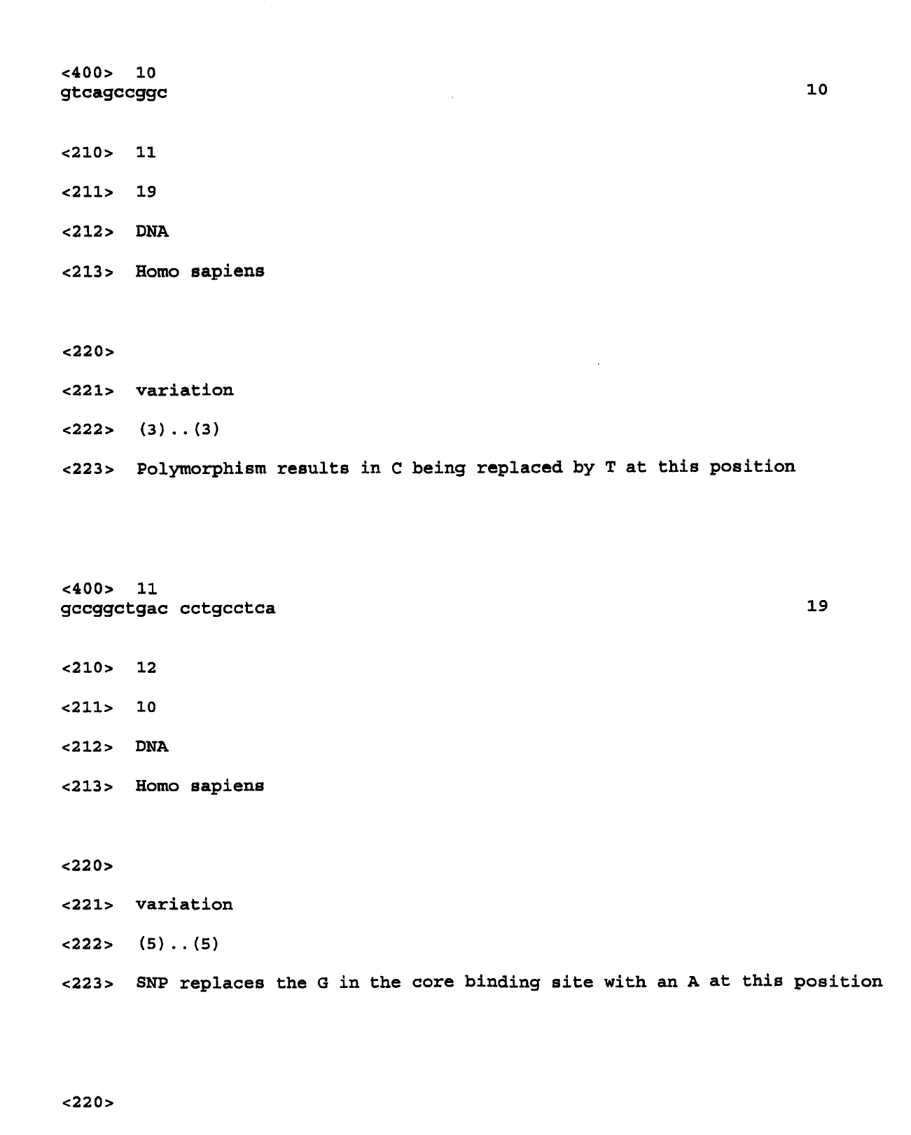

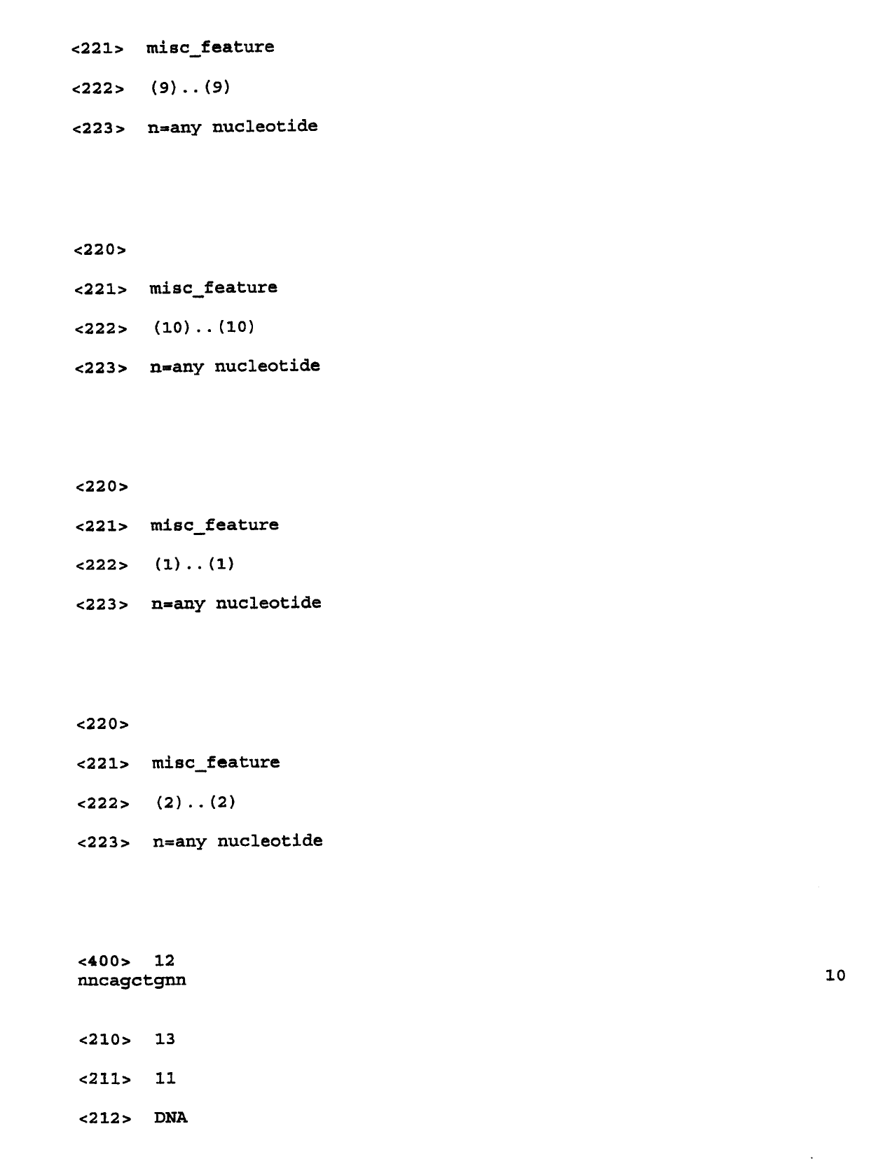

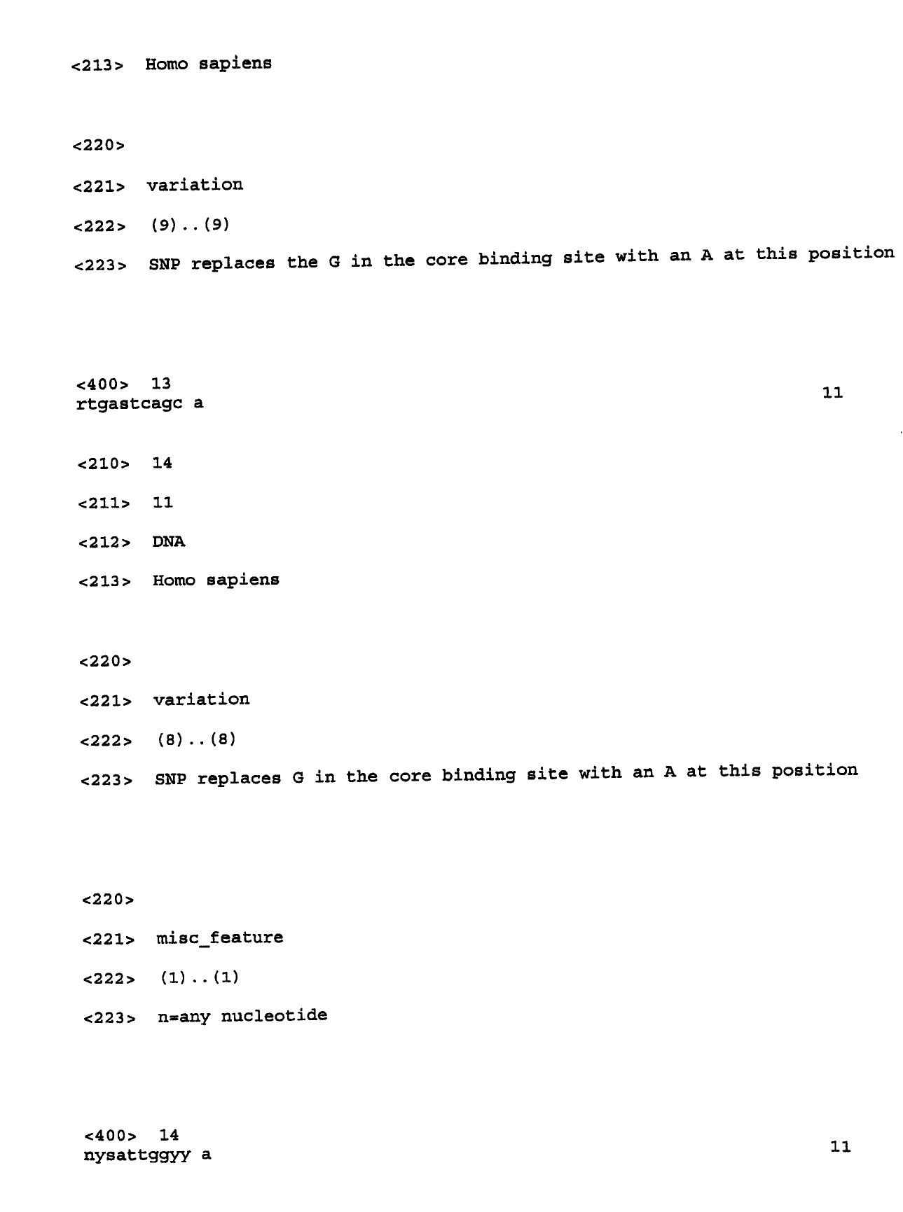

### DEFINITIONS

bp=base pair
kb=kilobase; 1000 base pairs
ecNOS=endothelial constitutive nitric oxide synthase
iNOS=inducible nitric oxide synthase
ESRD=end-stage renal disease
HTN=hypertension
NIDDM=noninsulin-dependent diabetes mellitus
CRF=chronic renal failure
T-GF=tubulo-glomerular feedback
CRG=compensatory renal growth
MODY=maturity-onset diabetes of the young
RFLP=restriction fragment length polymorphism
MASDA=multiplexed allele-specific diagnostic assay
MADGE=microtiter array diagonal gel electrophoresis
OLA=oligonucleotide ligation assay
DOL=dye-labeled oligonucleotide ligation assay
SNP=single nucleotide polymorphism
PCR=polymerase chain reaction

“Polynucleotide” and “oligonucleotide” are used interchangeably and mean a linear polymer of at least 2 nucleotides joined together by phosphodiester bonds and may consist of either ribonucleotides or deoxyribonucleotides.

“Sequence” means the linear order in which monomers occur in a polymer, for example, the order of amino acids in a polypeptide or the order of nucleotides in a polynucleotide.

“Polymorphism” refers to a set of genetic variants at a particular genetic locus among individuals in a population.

“Promoter” means a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) coding region, as well as intervening non-coding sequences (“introns”) between individual coding segments (“exons”). A promoter is herein considered as a part of the corresponding gene. Coding refers to the representation of amino acids, start and stop signals in a three base “triplet” code. Promoters are often upstream (“5′ to ”) the transcription initiation site of the gene.

“Gene therapy” means the introduction of a functional gene or genes from some source by any suitable method into a living cell to correct for a genetic defect.

“Reference allele” or “reference type” means the allele designated in the GenBank sequence listing for a given gene, in this case GenBank Accession Number AF032908 for the ecNOS gene.

“Genetic variant” or “variant” means a specific genetic variant which is present at a particular genetic locus in at least one individual in a population and that differs from the reference type.

As used herein the terms “patient” and “subject” are not limited to human beings, but are intended to include all vertebrate animals in addition to human beings.

As used herein the terms “genetic predisposition”, “genetic susceptibility” and “susceptibility” all refer to the likelihood that an individual subject will develop a particular disease, condition or disorder. For example, a subject with an increased susceptibility or predisposition will be more likely than average to develop a disease, while a subject with a decreased predisposition will be less likely than average to develop the disease. A genetic variant is associated with an altered susceptibility or predisposition if the allele frequency of the genetic variant in a population or subpopulation with a disease, condition or disorder varies from its allele frequency in the population without the disease, condition or disorder (control population) or a control sequence (reference type) by at least 1%, preferably by at least 2%, more preferably by at least 4% and more preferably still by at least 8%. Alternatively, an odds ratio of 1.5 was chosen as the threshold of significance based on the recommendation of Austin et al. in Epidemiol. Rev., 16:65-76, 1994. “[E]pidemiology in general and case-control studies in particular are not well suited for detecting weak associations (odds ratios <1.5).” Id. at 66.

As used herein “isolated nucleic acid” means a species of the invention that is the predominate species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

As used herein, “allele frequency” means the frequency that a given allele appears in a population.

### DETAILED DESCRIPTION OF THE PREFERRED EMBODIMENTS

All publications, patents, patent applications and other references cited in this application are herein incorporated by reference in their entirety as if each individual publication, patent, patent application or other reference were specifically and individually indicated to be incorporated by reference.

Nitric oxide (NO) has been strongly implicated in apoptosis of endothelial (Bonfoco et al., Proc. Natl. Acad. Sci. USA, 92:7162-7166, 1995) and vascular smooth muscle cells (Nishio et al., Biochem. Biophlys. Res. Commun., 221:163-168, 1996). Nitric oxide, which is vasodilatory, antagonizes the vasoconstrictive effects of angiotensin II and endothelins. Since angiotensin II promotes renal injury, nitric oxide may protect against renal injury from systemic disease such as hypertension and non-insulin dependent diabetes mellitus (NDDM; Bataineh and Raij, Kidney Int., 68:S140S19, 1998) Nitric oxide has also been implicated in the progression of renal disease in rats (Brooks and Contino, Pharmacology, 56:257-261, 1998) and humans (Noris and Remuzzi, Contrib. Nephrol. 119:8-15, 1996; Kone, Am. J. Kidney Dis., 30:311-333, 1997; Aiello et al., Kidney Int., 65:S63-S67, 1998; Raij, Hypertension, 31:189-193, 1998). The nitric oxide synthase genes are recognized candidate genes for hypertension, renal failure, and cardiovascular in general (Soubrier, Hypertension, 31:189-193, 1998)

NO can directly oxidize (and activate) thiol-containing proteins such as NF-κB and AP-1 (Stamler, Cell, 78:931-936, 1994). NO can either promote apoptosis or prevent it. Above a threshold concentration, NO seems to stimulate apoptosis (Bonfoco et al., Proc. Natl. Acad. Sci. USA, 92:7162-7166, 1995; Stamnler, Cell, 78:931-936, 1994).

The highest amount of NO is made by the inducible NO synthase (iNOS, NOS II), which is fully active at the prevailing intracellular calcium concentration (Caᵢ˜100 nM), and, once induced, remains active for days, producing nanomolar amounts of NO (Yu et al., Proc. Natl. Acad. Sci. USA, 91:1691-1695, 1994). The cis regulatory sequences for iNOS are not fully known. However, a region of 1798 nucleotides (nt) immediately upstream (5′) of the gene has been sequenced. Additional regulatory regions far upstream have been found in the human iNOS gene (de Vera ME et al., Proc. Natl. Acad. Sci. USA, 93:1054-1059, 1996), but have not yet been reported. Increased inducibility of iNOS would have conferred an important selection advantage, since iNOS is thought to be the major mechanism for immune cell-mediated killing of infectious agents such as parasites (e.g. malaria), bacteria, and viruses.

An additional source of renal NO is endothelial constitutive NOS (ecNOS, NOS III). ecNOS requires an elevation of Caᵢ to be active, since it must bind calmodulin for activity. ecNOS, which produces picomolar amounts of NO, may thus seem an unlikely source of large amounts of NO, but it is specifically activated by shear stress (Awolesi et al., Surgery, 116:439-445, 1994), and may be involved in arterial remodeling. Like adenosine and endothelin-1, ecNOS may therefore account for the clinical observation that the rate of progression of CRF is proportional to the degree of hypertension. Single nucleotide variations in the 5′ promoter region (1600 nt) of ecNOS might thus allow for increased induction.

Novel Polymorphisms

The human endothelial constitutive nitric oxide snythase (ecNOS, NOS3) gene promoter region resides on chromosome 7. The sequence of the ecNOS promoter has been published (GenBank accession # AF032908) (SEQ ID NO: 1). The present application provides 4 single nucleotide polymorphisms (SNPs) within the ecNOS promoter region. The location of these SNPs within the ecNOS promoter as well as the wild type and variant nucleotides are given in Table 25.

Preparation of Samples

The presence of genetic variants in the above gene or its control regions, or in any other genes that may affect susceptibility to disease is determined by screening nucleic acid sequences from a population of individuals for such variants. The population is preferably comprised of some individuals with the disease of interest, so that any genetic variants that are found can be correlated with disease. The population is also preferably comprised of some individuals that have known risk for the disease. The population should preferably be large enough to have a reasonable chance of finding individuals with the sought-after genetic variant. As the size of the population increases, the ability to find significant correlations between a particular genetic variant and susceptibility to disease also increases.

The nucleic acid sequence can be DNA or RNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or MRNA, the target nucleic acid must be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably is by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR)(see, Wu and Wallace, Genomics, 4:560-569, 1989; Landegren et al., Science, 241:1077-1080, 1988), transcription amplification (Kwoh et al., Proc. Natl. Acad. Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factors for the disease. The populations should preferably be large enough to have a reasonable chance to find correlations between a particular genetic variant and susceptibility to the disease of interest. In addition, the allele frequency of the genetic variant in a population or subpopulation with the disease or pathology should vary from its allele frequency in the population without the disease pathology (control population) or the control sequence (wild type) by at least 1%, preferably by at least 2%, more preferably by at least 4% and more preferably still by at least 8%.

Determination of unknown genetic variants, and in particular SNPs, within a particular nucleotide sequence among a population may be determined by any method known in the art, for example and without limitation, direct sequencing, restriction length fragment polymorphism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, ₃rd ed., Wiley, 1995 and Sambrook et al., Molecular Cloning, 2^{nd} ed., Chap. 13, Cold Spring Harbor Laboratory Press, 1989. Sequencing can be carried out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

RFLP analysis (see, e.g. U.S. Pat. Nos. 5,324,631 and 5,645,995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in <220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.). Double strands are first heat-denatured. The single strands are then subjected to polyacrylamide gel electrophoresis under non-denaturing conditions at constant temperature (i.e. low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled wild type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET)(Keen et al., Trends Genet. 7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM)is based on the chemical reactivity of thymine (T) when mismatched with cytosine, guanine or thymine and the chemical reactivity of cytosine(C) when mismatched with thymine, adenine or cytosine (Cotton et al., Proc. Natl. Acad. Sci. USA, 85:4397-4401, 1988). Duplex DNA formed by hybridization of a wild type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the wild type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998, and will only be briefly reviewed here.

One type of assay has been termed an array hybridization assay, an example of which is the multiplexed allele-specific diagnostic assay (MASDA) (U.S. Pat. No. 5,834,181; Shuber et al., Hum. Molec. Genet., 6:337-347, 1997). In MASDA, samples from multiplex PCR are immobilized on a solid support. A single hybridization is conducted with a pool of labeled allele specific oligonucleotides (ASO). Any ASO that hybridizes to the samples are removed from the pool of ASOs. The support is then washed to remove unhybridized ASOs remaining in the pool. Labeled ASO remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during the PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons assay, the ASO probes contain complementary sequences flanking the-target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can be achieved by Microtiter Array Diagonal Gel Electrophoresis (MADGE)(Day and Humphries, Anal. Biochem., 222:389-395, 1994). In this assay restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA)(Sommer et al., Mayo Clin. Proc., 64:1361-1372 1989; Sarker et al., Anal. Biochem. 1990), allele-specific amplification (ASA), allele-specific PCR, and amplification refractory mutation system (ARMS)(Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determine whether an individual is homozygous or heterozygous for the mutation (Dutton and Sommer, Bio Techniques, 11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers have a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can by used as a template.

In one method for mass screening for SNPs based on the OLA, amplified DNA templates are analyzed for their ability to serve as templates for ligation reactions between labeled oligonucleotide probes (Samotiaki et al., Genomics, 20:238-242, 1994). In this assay, two allele-specific probes labeled with either of two lanthanide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J. Hum. Genet., 52:46-59, 1993. In this method the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled dNTP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al., Human Mut., 7:346-354, 1996). In this method, incorporation of the ddNTP is determined using an automatic gel sequencer.

Minisequencing has also been adapted for use with microarrays (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Jan. 21, 1999. PCR products are spotted on the array and allowed to anneal. The extension (elongation) reaction is carried out using a polymerase, a labeled dNTP and noncompeting ddNTPs. Incorporation of the labeled DNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with minisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease. In this method, a biological sample is obtained from a subject. The subject can be a human being or any vertebrate animal. The biological sample must contain polynucleotides and preferably genomic DNA. Samples that do not contain genomic DNA, for example, pure samples of mammalian red blood cells, are not suitable for use in the method. The form of the polynucleotide is not critically important such that the use of DNA, cDNA, RNA or mRNA is contemplated within the scope of the method. The polynucleotide is then analyzed to detect the presence of a genetic variant where such variant is associated with an increased risk of developing a disease, condition or disorder, and in particular breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the genetic variant is located at one of the polymorphic sites contained in Table 25. In another embodiment, the genetic variant is one of the variants contained in Table 25 or the complement of any of the variants contained in Table 25. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1 or the complement of SEQ ID NO: 1. In one preferred embodiment, the sequence contains at least one polymorphic site associated with a disease, and in breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the polymorphic site is selected from the group contained in Table 25. In another embodiment, the polymorphic site contains a genetic variant, and in particular, the genetic variants contained in Table 25 or the complements of the variants in Table 25. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, and in particular breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemnic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. The kits contain, at a minimum, at least one polynucleotide of at least 10 contiguous nucleotides of SEQ ID NO: 1 or the complement of SEQ ID NO: 1. In one embodiment, the polynucleotide contains at least one polymorphic site, preferably a polymorphic site selected from the group contained in Table 25. Alternatively the 3′ end of the polynucleotide is immediately 5′ to a polymorphic site, preferably a polymorphic site contained in Table 25. In one embodiment, the polymorphic site contains a genetic variant, preferably a genetic variant selected from the group contained in Table 25. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder caused either directly or indirectly by the presence of one or more single nucleotide polymorphisms. In this method genetic material from a patient, for example, DNA, cDNA, RNA or MRNA is screened for the presence of one or more SNPs associated with the disease of interest. Depending on the type and location of the SNP, a treatment regime is designed to counteract the effect of the SNP. For example and without limitation, genetic material from a patient suffering from end-stage renal disease (ESRD) can be screened for the presence of SNPs associated with ESRD. If one or more of the SNPs found disrupt a sequence in the ecNOS promoter region, such that there is less nitric oxide (NO) produced in tissues such as endothelial cells, a treatment, such as oral administration of L-arginine, a substrate for nitric oxide production, is devised to counteract the decreased nitric oxide production due to the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art. Once designed, the gene can be placed in the individual by any suitable means known in the art. (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have an increased susceptibility to a disease, condition or disorder due to the presence of one or more single nucleotide polymorphisms. In this embodiment, genetic material, such as DNA, CDNA, RNA or MrRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

For example, and without limitation, a patient with an increased risk of developing renal disease due to the presence of a SNP in the ecNOS promoter could be given treatment to increase the production of nitric oxide (NO) by, for example the oral administration of L-arginine, thus reducing the risk of developing renal disease.

### EXAMPLES

Position of the single nucleotide polymorphism (SNP) is given according to the numbering scheme in GenBank Accession Number AF032908. Thus, all nucleotides will be positively numbered, rather than bear negative numbers reflecting their position upstream from the transcription initiation site, a scheme often used for promoters. The two numbering systems can be easily interconverted, if necessary. GenBank sequences can be found at http://www.ncbi.nlm.nih.gov/

In the following examples, SNPs are written as “reference sequence” (or “wild type”) “nucleotide”→“variant nucleotide.” Changes in nucleotide sequences are indicated in bold print. The standard nucleotide abbreviations are used in which A=adenine, C=cytosine, G-guanine, T=thymine, M-A or C, R-A or G, W=A or T, S=C or G, Y=C or T, K=G or T, V=A or C or G, H=A or C or T; D=A or G or T; B=C or G or T; N=A or C or G or T

### Example 1

### Amplification of ecNOS Promoter Genomic DNA

Leukocytes were obtained from human whole blood collected with EDTA as an anticoagulant. Blood was obtained from a group of black men, black women, white men, and white women without any known disease. Blood was also obtained from individuals breast cancer, lung cancer, prostate cancer, NIDDM, ESRD due to NIDDM, HTN, ESRD due to HTN, myocardial infarction, colon cancer, ASPVD due to HTN, CVA due to HTN, cataracts due to HTN, HTN CM, MI due to HTN, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder as indicated in the tables below.

Genomic DNA was purified from the collected leukocytes using standard protocols well known to those of ordinary skill in the art of molecular biology (Ausubel et al., Short Protocols in Molecular Biology, 3^{rd} ed, John Wiley & Sons, 1995; Sambrook et al., Molecular Cloning, Cold Spring Harbor Laboratory Press, 1989; and Davis et al., Basic Methods in Molecular Biology, Elsevier Science Publishing, 1986). DNA encoding the ecNOS promoter region was amplified by polymerase chain reaction (PCR). One hundred nanograms of purified genomic DNA was used in each PCR reaction.

Standard PCR reaction conditions were used. Methods for conducting PCR are well known in the art and can be found, for example, in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990. One set of primers were used to span the ecNOS promoter region. The sequence of the forward primer is 5′ GAG TCT GGC CAA CAC AAA TCC 3′. (SEQ ID NO: 2). The sequence of the reverse primer is 5′ CTC TAG GGT CAT GCA GGT TCT C 3′. (SEQ ID NO: 3). The PCR product spanned positions +2356 to +3010 of the ecNOS promoter

These primers were chosen to have a melting temperature (Tm) close to 59° C. For the information derived for the Group I Diseases, PCR was performed according to the following protocol: 4 min at 95° C.; 29 cycles, each consisting of 40 seconds denaturation at 95° C., 20 seconds annealing at 59° C., and 1 min extension at 73° C.; followed by final extension for 4 min at 73° C. For the information derived for the Group II Diseases, PCR was performed according to the following protocol: 5 min at 94° C.; 45 cycles, each consisting of 45 seconds denaturation at 94° C., 45 seconds annealing at 64° C., and 45 seconds extension at 72° C.; followed by final extension for 10 min at 72° C.

PCR product was purified on a Qiagen column to remove unreacted dNTPs, Taq polymerase, etc., and then subjected to cycle sequencing using a Perkin-Elmer dye terminator (BigDye), and the same primers as in the original PCR. Sequencing product was purified free of unincorporated dye by precipitation, and loaded onto a slab gel of an ABI 377 machine. Peaks were analyzed by eye for heterozygosity, as well as by the Sequencher software program. Gel traces were discarded if they did not meet strict criteria. Samples were run in uniplex fashion (one sample per lane). The information for the Group I Diseases was derived from genes sequenced through cycle sequencing.

The SNP typing for the Group II Diseases was accomplished through a method called pyrosequencing. Pyrosequencing is a method of sequencing DNA by synthesis, where the addition of one of the four dNTPs that correctly matches the complementary base on the template strand is detected. Detection occurs via utilization of the pyrophosphate molecules liberated upon the addition of bases to the elongating synthetic strand. The pyrophosphate molecules are used to make ATP, which in turn drives the emission of photons in a luciferin/luciferase reaction, and these photons are detected by the pyrosequencing instrument.

A Luc96 Pyrosequencer (Pyrosequencing AB, Uppsala, Sweden) was used under default operating conditions supplied by the manufacturer. Primers were designed to anneal within 5 bases of the polymorphism, to serve as sequencing primers. Patient genomic DNA was subject to PCR using amplifying primers that amplify an approximately 200 base pair amplicon containing the polymorphisms of interest. One of the amplifying primers, with orientation opposite to the sequencing primer, was biotinylated. This allowed the selection of a single stranded template for pyrosequencing, whose orientation was complementary to the sequencing primer.

Amplicons prepared from genomic DNA were isolated by binding to streptavidin-coated magnetic beads according to the manufacturer's protocol (Dynal, Oslo, Norway; US office: Lake Success, N.Y.). After denaturation in NaOH, the biotinylated strands were separated from their complementary strands using magnets. After washing the magnetic beads, the biotinylated template strands still bound to the beads were transferred to 96-well plates.

The sequencing primers were added, annealing was carried out at 95° for 2 minutes, and plates were placed in the Pyrosequencer. The enzymes, substrates and dNTPs used for synthesis and pyrophosphate detection were added to the instrument immediately prior to sequencing. The Luc96 software requires definition of a program of adding the four dNTPs that is specific for the location of the sequencing primer, the DNA composition flanking the SNP, and the two possible alleles at the polymorphic locus. This order of adding the bases generates theoretical outcomes of light intensity patterns for each of the two possible homozygous states and the single heterozygous state. The Luc96 software then compares the actual outcome to the theoretical outcome and calls a genotype for each well. Each sample is also assigned one of three confidence scores: pass, uncertain, fail. The results for each plate are outputted as a text file and processed in Excel using a Visual Basic program to generate a report of genotype and allele frequencies for the various disease and population cell groupings represented on the 96 well plate.

A summary of the polymorphisms detected can be found in Table 25.

### Example 2

### G to A Transition at Position 2548 of Human ecNOS Promoter

TABLE 1
ALLELE FREQUENCIES FOR GROUP I DISEASES

| | G | A |
|---|---|---|
| CONTROL | | |
| Black men (n = 84 chromosomes) | 10 (12%) | 74 (88%) |
| Black women (n = 74 chromosomes) | 18 (24%) | 56 (76%) |
| White men (n = 88 chromosomes) | 31 (35%) | 57 (65%) |
| White women (n = 106 chromosomes) | 35 (34%) | 71 (66%) |
| DISEASE | | |
| BREAST CANCER | | |
| Black women (n = 40 chromosomes) | 7 (18%) | 33 (82%) |
| White women (n = 38 chromosomes) | 12 (32%) | 26 (68%) |
| LUNG CANCER | | |
| Black men (n = 40 chromosomes) | 5 (13%) | 35 (87%) |
| Black women (n = 32 chromosomes) | 6 (19%) | 26 (81%) |
| White men (n = 40 chromosomes) | 17 (43%) | 23 (57%) |
| White women (n = 22 chromosomes) | 8 (36%) | 14 (64%) |
| PROSTATE CANCER | | |
| Black men (n = 40 chromosomes) | 12 (30%) | 28 (70%) |
| White men (n = 40 chromosomes) | 18 (45%) | 22 (55%) |
| NIDDM | | |
| Black men (n = 4 chromosomes) | 1 (25%) | 3 (75%) |
| Black women (n = 6 chromosomes) | 1 (17%) | 5 (83%) |
| White men (n = 8 chromosomes) | 0 (0%) | 8 (100%) |
| White women (n = 20 chromosomes) | 5 (25%) | 15 (75%) |
| ESRD due to NIDDM | | |
| Black men (n = 12 chromosomes) | 1 (8%) | 11 (92%) |
| Black women (n = 16 chromosomes) | 2 (13%) | 14 (88%) |
| White men (n = 10 chromosomes) | 2 (20%) | 8 (80%) |
| White women (n = 8 chromosomes) | 2 (25%) | 6 (75%) |
| HYPERTENSION (HTN) | | |
| Black men (n = 24 chromosomes) | 3 (13%) | 21 (87%) |
| Black women (n = 24 chromosomes) | 2 (8%) | 22 (92%) |
| White men (n = 22 chromosomes) | 7 (32%) | 15 (68%) |
| White women (n = 20 chromosomes) | 8 (40%) | 12 (60%) |
| ESRD due to HTN | | |
| Black men (n = 20 chromosomes) | 4 (20%) | 16 (80%) |
| Black women (n = 18 chromosomes) | 0 (0%) | 18(100%) |
| White men (n = 18 chromosomes) | 5 (28%) | 13 (72%) |
| White women (n = 18 chromosomes) | 3 (17%) | 15 (83%) |
| MYOCARDIAL INFARCTION | | |
| White women (n = 16 chromosomes) | 5 (31%) | 11 (69%) |

TABLE 2
ALLELE FREQUENCY FOR GROUP II DISEASES

| Disease | Race | CHROMOSOMES | N | G | N | A |
|---|---|---|---|---|---|---|
| Controls | African-American | 90 | 16 | 17.8% | 74 | 82.2% |
| | Caucasian | 94 | 33 | 35.1% | 61 | 64.9% |
| Colon cancer | African-American | 48 | 7 | 14.6% | 41 | 85.4% |
| | Caucasian | 44 | 11 | 25.0% | 33 | 75.0% |
| Hypertension | African-American | 46 | 4 | 8.7% | 42 | 91.3% |
| ASPVD due to HTN | African-American | 54 | 4 | 7.4% | 50 | 92.6% |
| | Caucasian | 50 | 13 | 26.0% | 37 | 74.0% |
| CVA due to HTN | African-American | 48 | 9 | 18.8% | 39 | 81.3% |
| | Caucasian | 48 | 16 | 33.3% | 32 | 66.7% |
| Cataracts due to HTN | African-American | 48 | 4 | 8.3% | 44 | 91.7% |
| | Caucasian | 44 | 11 | 25.0% | 33 | 75.0% |
| HTN CM | African-American | 48 | 9 | 18.8% | 39 | 81.3% |
| MI due to HTN | African-American | 42 | 6 | 14.3% | 36 | 85.7% |
| | Caucasian | 46 | 13 | 28.3% | 33 | 71.7% |
| NIDDM | African-American | 48 | 2 | 4.2% | 46 | 95.8% |
| | Caucasian | 48 | 12 | 25.0% | 36 | 75.0% |
| ASPVD due to NIDDM | African-American | 48 | 5 | 10.4% | 43 | 89.6% |
| | Caucasian | 48 | 9 | 18.8% | 39 | 81.3% |
| CVA due to NIDDM | African-American | 48 | 5 | 10.4% | 43 | 89.6% |
| | Caucasian | 48 | 18 | 37.5% | 30 | 62.5% |
| Ischemic CM | African-American | 48 | 13 | 27.1% | 35 | 72.9% |
| Ischemic CM with NIDDM | African-American | 48 | 4 | 8.3% | 44 | 91.7% |
| MI due to NIDDM | African-American | 46 | 4 | 8.7% | 42 | 91.3% |
| | Caucasian | 48 | 22 | 45.8% | 26 | 54.2% |
| Afib without valvular disease | African-American | 48 | 8 | 16.7% | 40 | 83.3% |
| | Caucasian | 48 | 20 | 41.7% | 28 | 58.3% |
| Anxiety | African-American | 48 | 9 | 18.8% | 39 | 81.3% |
| Asthma | African-American | 46 | 6 | 13.0% | 40 | 87.0% |
| | Caucasian | 48 | 22 | 45.8% | 26 | 54.2% |
| COPD | African-American | 48 | 12 | 25.0% | 36 | 75.0% |
| | Caucasian | 48 | 16 | 33.3% | 32 | 66.7% |
| Cholecystectomy | African-American | 48 | 6 | 12.5% | 42 | 87.5% |
| | Caucasian | 48 | 19 | 39.6% | 29 | 60.4% |
| DJD | African-American | 48 | 5 | 10.4% | 43 | 89.6% |
| ESRD and frequent de-clots | African-American | 48 | 8 | 16.7% | 40 | 83.3% |
| | Caucasian | 44 | 12 | 27.3% | 32 | 72.7% |
| ESRD due to FSGS | African-American | 48 | 7 | 14.6% | 41 | 85.4% |
| | Caucasian | 44 | 9 | 20.5% | 35 | 79.5% |
| ESRD due to IDDM | African-American | 48 | 4 | 8.3% | 44 | 91.7% |
| Seizure disorder | African-American | 44 | 5 | 11.4% | 39 | 88.6% |
| | Caucasian | 48 | 14 | 29.2% | 34 | 70.8% |

TABLE 3
GENOTYPE FREQUENCIES FOR GROUP I DISEASES

| | G/G | G/A | A/A |
|---|---|---|---|
| CONTROLS | | | |
| Black men (n = 42) | 0 (0%) | 10 (24%) | 32 (76%) |
| Black women (n = 37) | 2 (5%) | 14 (38%) | 21 (57%) |
| White men (n = 44) | 6 (14%) | 19 (43%) | 19 (43%) |
| White women (n = 53) | 3 (6%) | 29 (55%) | 21 (40%) |
| DISEASE | | | |
| BREAST CANCER | | | |
| Black women (n = 20) | 0 (0%) | 7 (35%) | 13 (65%) |
| White women (n = 19) | 1 (5%) | 10 (53%) | 8 (42%) |
| LUNG CANCER | | | |
| Black men (n = 20) | 0 (0%) | 5 (25%) | 15 (75%) |
| Black women (n = 16) | 0 (0%) | 6 (38%) | 10 (62%) |
| White men (n = 20) | 2 (10%) | 13 (65%) | 5 (25%) |
| White women (n = 11) | 2 (18%) | 4 (36%) | 5 (45%) |
| PROSTATE CANCER | | | |
| Black men (n = 20) | 1 (5%) | 10 (50%) | 9 (45%) |
| White men (n = 20) | 2 (10%) | 14 (70%) | 4 (20%) |
| NIDDM | | | |
| Black men (n = 2) | 0 (0%) | 1 (50%) | 1 (50%) |
| Black women (n = 3) | 0 (0%) | 1 (33%) | 2 (67%) |
| White men (n = 4) | 0 (0%) | 0 (0%) | 4 (100%) |
| White women (n = 10) | 0 (0%) | 5 (50%) | 5 (50%) |
| ESRD due to NIDDM | | | |
| Black men (n = 7) | 0 (0%) | 7 (100%) | 0 (0%) |
| Black women (n = 7) | 1 (14%) | 4 (57%) | 2 (29%) |
| White men (n = 7) | 1 (14%) | 5 (71%) | 1 (14%) |
| White women (n = 3) | 0 (0%) | 3(100%) | 0 (0%) |
| HYPERTENSION (HTN) | | | |
| Black men (n = 12) | 0 (0%) | 3 (25%) | 9 (75%) |
| Black women (n = 12) | 0 (0%) | 2 (17%) | 10 (83%) |
| White men (n = 11) | 1 (9%) | 5 (45%) | 5 (45%) |
| White women (n = 10) | 1 (10%) | 6 (60%) | 3 (30%) |
| ESRD due to HTN | | | |
| Black men (n = 10) | 1 (10%) | 2 (20%) | 7 (70%) |
| Black women (n = 9) | 0 (0%) | 0 (0%) | 9 (100%) |
| White men (n = 9) | 0 (0%) | 5 (56%) | 4 (44%) |
| White women (n = 9) | 0 (0%) | 3 (33%) | 6 (67%) |
| MYOCARDIAL INFARCTION | | | |
| White women (n = 6) | 3 (50%) | 3 (50%) | 0 (0%) |

TABLE 4
GENOTYPE FREQUENCY FOR GROUP II DISEASES

| Disease | Race | People | N | G/G | N | G/A | N | A/A |
|---|---|---|---|---|---|---|---|---|
| Controls | African American | 45 | 2 | 4.4% | 12 | 26.7% | 31 | 68.9% |
| | Caucasian | 47 | 5 | 10.6% | 23 | 48.9% | 19 | 40.4% |
| Colon cancer | African-American | 24 | 0 | 0.0% | 7 | 29.2% | 17 | 70.8% |
| | Caucasian | 22 | 1 | 4.5% | 9 | 40.9% | 12 | 54.5% |
| Hypertension | African-American | 23 | 0 | 0.0% | 4 | 17.4% | 19 | 82.6% |
| ASPVD due to HTN | African-American | 27 | 0 | 0.0% | 4 | 14.8% | 23 | 85.2% |
| | Caucasian | 25 | 1 | 4.0% | 11 | 44.0% | 13 | 52.0% |
| CVA due to HTN | African-American | 24 | 1 | 4.2% | 7 | 29.2% | 16 | 66.7% |
| | Caucasian | 24 | 3 | 12.5% | 10 | 41.7% | 11 | 45.8% |
| Cataracts due to HTN | African-American | 24 | 0 | 0.0% | 4 | 16.7% | 20 | 83.3% |
| | Caucasian | 22 | 1 | 4.5% | 9 | 40.9% | 12 | 54.5% |
| HTN CM | African-American | 24 | 0 | 0.0% | 9 | 37.5% | 15 | 62.5% |
| NIDDM | African-American | 24 | 0 | 0.0% | 2 | 8.3% | 22 | 91.7% |
| | Caucasian | 24 | 0 | 0.0% | 12 | 50.0% | 12 | 50.0% |
| ASPVD due to NIDDM | African-American | 24 | 0 | 0.0% | 5 | 20.8% | 19 | 79.2% |
| | Caucasian | 24 | 1 | 4.2% | 7 | 29.2% | 16 | 66.7% |
| CVA due to NIDDM | African-American | 24 | 0 | 0.0% | 5 | 20.8% | 19 | 79.2% |
| | Caucasian | 24 | 4 | 16.7% | 10 | 41.7% | 10 | 41.7% |
| Ischemic CM | African-American | 24 | 4 | 16.7% | 5 | 20.8% | 15 | 62.5% |
| Ischemic CM with NIDDM | African-American | 24 | 0 | 0.0% | 4 | 16.7% | 20 | 83.3% |
| Afib without valvular disease | African-American | 24 | 2 | 8.3% | 4 | 16.7% | 18 | 75.0% |
| | Caucasian | 24 | 4 | 16.7% | 12 | 50.0% | 8 | 33.3% |
| Anxiety | African-American | 24 | 1 | 4.2% | 7 | 29.2% | 16 | 66.7% |
| Asthma | African-American | 23 | 0 | 0.0% | 6 | 26.1% | 17 | 73.9% |
| | Caucasian | 24 | 5 | 20.8% | 12 | 50.0% | 7 | 29.2% |
| COPD | African-American | 24 | 2 | 8.3% | 8 | 33.3% | 14 | 58.3% |
| | Caucasian | 24 | 3 | 12.5% | 10 | 41.7% | 11 | 45.8% |
| Cholecystectomy | African-American | 24 | 0 | 0.0% | 6 | 25.0% | 18 | 75.0% |
| | Caucasian | 24 | 4 | 16.7% | 11 | 45.8% | 9 | 37.5% |
| DJD | African-American | 24 | 0 | 0.0% | 5 | 20.8% | 19 | 79.2% |
| ESRD and frequent de-clots | African-American | 24 | 3 | 12.5% | 2 | 8.3% | 19 | 79.2% |
| | Caucasian | 22 | 2 | 9.1% | 8 | 36.4% | 12 | 54.5% |
| ESRD due to FSGS | African-American | 24 | 0 | 0.0% | 7 | 29.2% | 17 | 70.8% |
| | Caucasian | 22 | 1 | 4.5% | 7 | 31.8% | 14 | 63.6% |
| ESRD due to IDDM | African-American | 24 | 0 | 0.0% | 4 | 16.7% | 20 | 83.3% |
| Seizure disorder | African-American | 22 | 0 | 0.0% | 5 | 22.7% | 17 | 77.3% |
| | Caucasian | 24 | 1 | 4.2% | 12 | 50.0% | 11 | 45.8% |

Allele-Specific Odds Ratios

The susceptibility allele is indicated, as well as the odds ratio (OR). Haldane's zero cell correction was used. If the odds ratio (OR) was ≧1.5, the 95% confidence interval (C.I.) is also given. An odds ratio of 1.5 was chosen as the threshold of significance based on the recommendation of Austin H et al. in Epidemiol. Rev., 16:65-76, (1994). “[E]pidemiology in general and case-control studies in particular are not well suited for detecting weak associations (odds ratios <1.5).” Id. at 66.

An example of the allele-specific odds ratio calculation is given below:

Colon Cancer: African-Americans

| | Cases | Controls |
|---|---|---|
| A | 41 | 74 |
| G | 7 | 16 |

The odds ratio is (41)(16)/(7)(74)=1.3. Therefore, African-Americans with the A allele have a 1.3 fold higher risk of developing colon cancer than African-Americans without the A allele. Odds ratios of 1.5 or higher are high-lighted below.

TABLE 5
ALLELE-SPECIFIC ODDS RATIOS FOR GROUP I DISEASES

| | SUSCEPTIBILITY | | |
|---|---|---|---|
| DISEASE | ALLELE | OR | 95% C.I. |
| Breast Cancer | | | |
| Black women | A | 1.5 | 0.6-4.0 |
| White women | G | 0.9 | |
| Lung Cancer | | | |
| Black men | G | 1.1 | |
| Black women | A | 1.4 | |
| White men | G | 1.4 | |
| White women | G | 1.2 | |
| Prostate Cancer | | | |
| Black men | G | 3.2 | 1.2-8.2 |
| White men | G | 1.5 | 0.7-3.2 |
| NIDDM | | | |
| Black men | G | 2.5 | 0.2-26.1 |
| Black women | A | 1.6 | 0.2-15 |
| White men | A | 9.3 | 1.2-72 |
| White women | A | 1.5 | 0.5-4.4 |
| ESRD due to NIDDM* | | | |
| Black men | A | 3.7 | 0.2-78 |
| Black women | A | 1.4 | |
| White men | G | 5.0 | 0.5-47 |
| White women | A | 1.0 | |
| Hypertension (HTN) | | | |
| Black men | G | 1.0 | |
| Black women | A | 3.5 | 0.8-17 |
| White men | A | 1.2 | |
| White women | G | 1.4 | |
| ESRD due to HTN*¹ | | | |
| Black men | G | 1.8 | 0.3-9.0 |
| Black women | A | 4.1 | 0.5-37 |
| White men | A | 0.8 | |
| White women | A | 3.3 | 0.7-15 |
| Myocardial Infarction | | | |
| White women | A | 1.1 | |

*Compared to group with NIDDM alone.
*¹Compared to group with HTN alone.

TABLE 6
ALLELE-SPECIFIC ODDS RATIOS FOR GROUP II DISEASES

| | | | | Lower | Upper | |
|---|---|---|---|---|---|---|
| | | | | Limit | Limit | |
| | | Risk | Odds | 95% | 95% | |
| Disease | Race | Allele | Ratio | CI | CI | Haldane |
| Colon cancer | African-American | A | 1.3 | 0.5 | 3.3 | |
| | Caucasian | A | 1.6 | 0.7 | 3.6 | |
| Hypertension | African-American | A | 2.3 | 0.7 | 7.2 | |
| ASPVD due to HTN* | African-American | A | 1.2 | 0.3 | 5.1 | |
| CVA due to HTN* | African-American | G | 2.4 | 0.7 | 8.5 | |
| Cataracts due to HTN* | African-American | A | 2.4 | 0.7 | 7.6 | |
| | Caucasian | A | 1.6 | 0.7 | 3.6 | |
| NIDDM | African-American | A | 5.0 | 1.1 | 22.6 | |
| | Caucasian | A | 1.6 | 0.7 | 3.5 | |
| ASPVD due to NIDDM*¹ | African-American | G | 2.7 | 0.5 | 14.5 | |
| | Caucasian | A | 1.4 | 0.5 | 3.8 | |
| CVA due to NIDDM*¹ | African-American | G | 2.7 | 0.5 | 14.5 | |
| | Caucasian | G | 1.8 | 0.7 | 4.3 | |
| Afib without valvular disease | African-American | A | 1.1 | 0.4 | 2.7 | |
| | Caucasian | G | 1.3 | 0.6 | 2.7 | |
| Anxiety | African-American | G | 1.1 | 0.4 | 2.6 | |
| Asthma | African-American | A | 1.4 | 0.5 | 4.0 | |
| | Caucasian | G | 1.6 | 0.8 | 3.2 | |
| COPD | African-American | G | 1.5 | 0.7 | 3.6 | |
| | Caucasian | A | 1.1 | 0.5 | 2.3 | |
| Cholecystectomy | African-American | A | 1.5 | 0.6 | 4.2 | |
| | Caucasian | G | 1.2 | 0.6 | 2.5 | |
| DJD | African-American | A | 1.9 | 0.6 | 5.4 | |
| ESRD and frequent de-clots | African-American | A | 1.1 | 0.4 | 2.7 | |
| | Caucasian | A | 1.4 | 0.7 | 3.2 | |
| ESRD due to FSGS | African-American | A | 1.3 | 0.5 | 3.3 | |
| | Caucasian | A | 2.1 | 0.9 | 4.9 | |
| ESRD due to IDDM | African-American | A | 2.4 | 0.7 | 7.6 | |
| Seizure disorder | African-American | A | 1.7 | 0.6 | 4.9 | |
| | Caucasian | A | 1.3 | 0.6 | 2.8 | |

*Compared to HTN alone.
*¹Compared to NIDDM alone.

Genotype-Specific Odds Ratios

The susceptibility allele (S) is indicated; the alternative allele at this locus is defined as the protective allele (P). Also presented is the odds ratio (OR) for each genotype (SS, SP; the odds ratio for the PP genotype is 1, since it is the reference group, and is not presented separately). For odds ratios ≧1.5, the 95% confidence interval (C.I.) is also given, in parentheses. An odds ratio of 1.5 was chosen as the threshold of significance based on the recommendation of Austin et al. in Epidemiol. Rel., 16:65-76, (1994). “[E]pidemiology in general and case-control studies in particular are not well suited for detecting weak associations (odds ratios <1.5).” Id. at 66.

Where Haldane's zero cell correction was employed, the odds ratio is so indicated with a superscript “H”. To minimize confusion, genotype-specific odds ratios are presented only for diseases in which the allele-specific odds ratio was at least 1.5.

An example is worked below, assuming that A is the susceptibility allele (S), and G is the protective allele (P).

African-American: ASPVD due to HTN (Compared to African-Americans with Hypertension)

| | Cases | Controls |
|---|---|---|
| AA (SS) | 23 | 19 |
| AG (SP) | 4 | 4 |
| GG (PP) | 0 | 0 |

Applying Haldane's correction, the above 2×3 table becomes:

African-American: ASPVD due to HTN (Compared to African-Americans with Hypertension)

| | Cases | Controls | Odds Ratios |
|---|---|---|---|
| CC (SS) | 47 | 39 | (47)(1)/(1)(39) = 1.2 |
| CT (SP) | 9 | 9 | (9)(1)/(1)(9) = 1.0 |
| TT (PP) | 1 | 1 | 1.0 (by definition) |

The odds ratios for individual genotypes are given below. Odds ratios of 1.5 or more are given below.

TABLE 7
GENOTYPE-SPECIFIC ODDS RATIOS FOR GROUP I DISEASES

| | SUSCEPTIBILITY | | |
|---|---|---|---|
| DISEASE | ALLELE | OR(SS) | OR(SP) |
| Breast Cancer | | | |
| Black women | A | 3.1 (0.3-28) | 2.6 (0.3-24) |
| Prostate Cancer | | | |
| Black men | G | 10.3 (1.0-105) | 3.4 (1.5-7.6) |
| White men | G | 1.6 (0.2-11) | 3.5 (1.0-12.6) |
| NIDDM | | | |
| Black men | G | 21.7 (1.1-437) | 3.1 (0.6-17) |
| Black women | G | 1.7 (0.2-18) | 0.9 |
| White men | A | 3.0 (0.3-26) | 0.3 (0-5.7) |
| White women | A | 1.8 (0.2-16) | 1.3 |
| ESRD due | | | |
| to NIDDM* | | | |
| Black men | G | 3.0 (0.1-108) | 15 (1.1-198) |
| White men | G | 9.0 (0.7-123) | 33 (2.9-374) |
| Hypertension | | | |
| (HTN) | | | |
| Black women | A | 2.4 (0.3-22) | 0.9 |
| ESRD due | | | |
| to HTN*¹ | | | |
| Black men | G | 3.8 (0.4-40) | 0.9 |
| Black women | A | 0.9 | 0.2 |
| White men | A | 2.5 (0.2-28) | 3.0 (0.3-34) |
| White women | A | 5.6 (0.5-64) | 1.6 (0.1-19) |

*Compared to group with NIDDM.
*¹Compared to group with HTN.

TABLE 8
GENOTYPE-SPECIFIC ODDS RATIOS FOR GROUP II DISEASES

| | | RISK | SS | | SP | |
|---|---|---|---|---|---|---|
| Disease | Race | ALLELE | O.R. | Haldane | O.R. | Haldane |
| Colon cancer | African-American | A | 0.0 | | 1.1 | |
| | Caucasian | A | 0.3 | | 0.6 | |
| Hypertension | African-American | A | 0.0 | | 0.5 | |
| ASPVD due to HTN* | African-American | A | 0.8 | H | 0.8 | |
| CVA due to HTN* | African-American | G | 3.5 | H | 2.1 | |
| Cataracts due to HTN* | African-American | A | 0.0 | | 0.5 | |
| | Caucasian | A | 0.3 | | 0.6 | |
| NIDDM | African-American | A | 0.0 | | 0.2 | |
| | Caucasian | A | 0.0 | | 0.8 | |
| ASPVD due to NIDDM*¹ | African-American | G | 1.2 | H | 2.9 | |
| | Caucasian | A | 2.3 | H | 0.4 | |
| CVA due to NIDDM*¹ | African-American | G | 1.2 | H | 2.9 | |
| | Caucasian | G | 10.7 | H | 1.0 | |
| Afib without valvular | African-American | A | 1.7 | | 0.6 | |
| disease | Caucasian | G | 1.9 | | 1.2 | |
| Anxiety | African-American | G | 1.0 | | 1.1 | |
| Asthma | African-American | A | 0.0 | | 0.9 | |
| | Caucasian | G | 2.7 | | 1.4 | |
| COPD | African-American | G | 2.2 | | 1.5 | |
| | Caucasian | A | 1.0 | | 0.8 | |
| Cholecystectomy | African-American | A | 0.0 | | 0.9 | |
| | Caucasian | G | 1.7 | | 1.0 | |
| DJD | African-American | A | 0.0 | | 0.7 | |
| ESRD and frequent de- | African-American | A | 2.4 | | 0.3 | |
| clots | Caucasian | A | 0.6 | | 0.6 | |
| ESRD due to FSGS | African-American | A | 0.0 | | 1.1 | |
| | Caucasian | A | 0.3 | | 0.4 | |
| ESRD due to IDDM | African-American | A | 0.0 | | 0.5 | |
| Seizure disorder | African-American | A | 0.0 | | 0.8 | |
| | Caucasian | A | 0.3 | | 0.9 | |

*Compared to HTN alone.
*¹Compared to NIDDM alone.

PCR and sequencing were conducted as in Example 1. The primers used were the same as in Example 1. The control samples are in good agreement with Hardy-Weinberg equilibrium, as follows:

In the control group for the Group I Diseases, a frequency of 0.12 for the G allele (“p”) and 0.88 for the A allele (“q”) among black male control individuals predicts genotype frequencies of 1% G/G, 22% G/A, and 77% A/A at Hardy-Weinberg equilibrium (p²+2pq+q²=1). The observed genotype frequencies were 0% G/G, 24% G/A, and 76% A/A, in excellent agreement with those predicted for Hardy-Weinberg equilibrium.

A frequency of 0.24 for the G allele (“p”) and 0.76 for the A allele (“q”) among black female control individuals predicts genotype frequencies of 6% G/G, 36% G/A, and 58% A/A at Hardy-Weinberg equilibrium (p²+2pq+q²=1). The observed genotype frequencies were 5% G/G, 38% G/A, and 57% A/A, in excellent agreement with those predicted for Hardy-Weinberg equilibrium.

A frequency of 0.35 for the G allele (“p”) and 0.65 for the A allele (“q”) among white male control individuals predicts genotype frequencies of 12% G/G, 46% G/A, and 42% A/A at Hardy-Weinberg equilibrium (p²+2pq+q²=1). The observed genotype frequencies were 14% G/G, 43% G/A, and 43% A/A, in very close agreement with those predicted for Hardy-Weinberg equilibrium.

A frequency of 0.34 for the G allele (“p”) and 0.66 for the A allele (“q”) among white female control individuals predicts genotype frequencies of 12% G/G, 38% G/A, and 50% A/A at Hardy-Weinberg equilibrium (p²+2pq+q²=1). The observed genotype frequencies were 6% G/G, 55% G/A, and 40% A/A, in fair agreement with those predicted for Hardy-Weinberg equilibrium.

In the control group for the Group II Diseases, a frequency of 0.18 for the G allele (“p”) and 0.82 for the A allele (“q”) among African-American control individuals predicts genotype frequencies of 3.2% G/G, 29.5% G/A, and 67.2% A/A at Hardy-Weinberg equilibrium (p²+2pq+q²=1). The observed genotype frequencies were 4.4% G/G, 26.7% G/A, and 68.9% A/A, in good agreement with those predicted for Hardy-Weinberg equilibrium.

A frequency of 0.35 for the G allele (“p”) and 0.65 for the A allele (“q”) among Caucasian control individuals predicts genotype frequencies of 12.25% G/G, 45.5% G/A, and 42.25% A/A at Hardy-Weinberg equilibrium (p²+2pq+q²=1). The observed genotype frequencies were 10.6% G/G, 48.9% G/A, and 40.4% A/A, in excellent agreement with those predicted for Hardy-Weinberg equilibrium.

Results

Using an allele-specific odds ratio of 1.5 or greater as a practical level of significance, the following observations can be made.

The odds ratio for the A allele at this locus was 1.5 (95% CI, 0.6-4.0) among black women with breast cancer. The odds ratio for the AG heterozygote was 2.6 (95% CI, 0.3-24), and was 3.1 (95% CI, 0.3-28) for the AA homozygote, indicating a dose-dependent increase in relative risk of disease. These data suggest that the A allele behaves as a dominant susceptibility allele.

The odds ratio for the G allele at this locus was 3.2 (95% CI, 1.2-8.2) for black men with prostate cancer. The odds ratio for the GA heterozygote was 3.4 (95% CI, 1.5-7.6), and was 10.3 (95% CI, 1.0-105) for the GG homozygote, indicating a dose-dependent increase in the relative risk of disease with two rather than one G allele. These data suggest that the G allele behaves as a dominant susceptibility allele with interaction on a multiplicative model [(10.3)˜(3.4)(3.4)].

The odds ratio for the G allele at this locus was 1.5 (95% CI, 0.7-3.2) for white men with prostate cancer. The odds ratio for the GA heterozygote was 3.5 (95% CI, 1.0-12.6), but for the GG homozygote was only 1.6 (95% CI, 0.2-11). In other words, there was approximately twice as high a relative risk of disease with only one allele as with two, suggesting that the G allele behaves as a co-dominant allele.

The odds ratio for the A allele at this locus was 1.6 (95% CI, 0.2-15) for black women with NIDDM. The genotype-specific odds ratios are not helpful, since they suggest that the G allele, rather than the A allele, is the susceptibility allele.

The odds ratio for the A allele at this locus was 9.3 (95% CI, 1.2-72) for white men with NIDDM. The odds ratio for the AG heterozygote [0.3 (95% CI, 0-5.7)] was actually less than 1, whereas the odds ratio for the AA homozygote was 3.0 (95% CI, 0.3-26). These data suggest that the A allele behaves in a recessive fashion.

The odds ratio for the A allele at this locus was 1.5 (95% CI, 0.5-4.4) among white women with NIDDM. The odds ratio for the AG heterozygote was 1.3, and for the AA homozygote was 1.8 (95% CI, 0.2-16), indicating a dose-dependent increase in relative risk of disease. These data suggest that the A allele behaves as a recessive susceptibility allele.

The odds ratio for the A allele at this locus was 3.7 (95% CI, 0.2-78) for black men with ESRD due to NIDDM, when compared to black men with NIDDM alone. The genotype-specific odds ratios are not helpful, since they suggest that the G allele, rather than the A allele, is the susceptibility allele.

The odds ratio for the G allele at this locus was 5.0 (95% CI, 0.5-47) for white men with ESRD due to NIDDM, when compared to white men with NIDDM alone. The odds ratio for the GA heterozygote was a remarkable 33 (95% CI, 2.9-374), but for the GG homozygote was only 9.0 (95% CI, 0.7-123). In other words, carrying only one susceptibility allele is associated with nearly four times the relative risk of disease as carrying two alleles. These data suggest that the G allele behaves in a co-dominant fashion.

The odds ratio for the A allele at this locus was 3.5 (95% CI, 0.8-17) among black women with hypertension (HTN). The odds ratio for the AG heterozygote was 0.9, and for the AA homozygote was 2.4 (95% CI, 0.3-22). These data suggest that the A allele behaves as a classical recessive susceptibility allele.

The odds ratio for the G allele at this locus was 1.8 (95% CI, 0.3-9.0) among black men with ESRD due to hypertension (HTN), when compared to black men with HTN alone. The odds ratio for the GA heterozygote was 0.9, and for the GG homozygote was 3.8 (95% CI, 0.4-40). These data suggest that the G allele behaves as a recessive disease-susceptibility allele.

The odds ratio for the A allele at this locus was 4.1 (95% CI, 0.5-37) among black women with ESRD due to HTN, when compared to black women with HTN alone. The genotype-specific odds ratios are unhelpful because they are less than one.

The odds ratio for the A allele at this locus was 3.3 (95% CI, 0.7-15) for white women with ESRD due to hypertension (HTN), when compared to white women with HTN alone. The odds ratio for the AG heterozygote was 1.6 (95% CI, 0.1-19), and for the AA homozygote was 5.6 (95% CI, 0.5-64), indicating a dose-dependent increase in the relative risk of disease with two rather than one G allele. These data suggest that the A allele behaves as a dominant susceptibility allele with interaction on more than a multiplicative model [5.6>(1.6)(1.6)].

For African-Americans with ASPVD due to NIDDM the odds ratio for the G allele was 2.7 (95% CI, 0.5-14.5), compared to African-Americans with NIDDM only. The odds ratio for the homozygote (G/G) was 1.2^{H} (95% CI, 0-143.2),while the odds ratio for the heterozygote (G/A) was 2.9 (95% CI, 0.5-16.7). These data suggest that the G allele acts in a co-dominant manner in this patient population. These data further suggest that the ecNOS gene is significantly associated with ASPVD due to NIDDM in African-Americans, i.e. abnormal activity of the ecNOS gene predisposes African-Americans to ASPVD due to NIDDM.

For Caucasians with asthma the odds ratio for the G allele was 1.6 (95% CI, 0.8-3.2). The odds ratio for the homozygote (GIG) was 2.7 (95% CI, 0.6-12.3), while the odds ratio for the heterozygote (G/A) was 1.4 (95% CI,0.5-4.3). These data suggest that the G allele acts in a recessive manner in this patient population. These data further suggest that the ecNOS gene is significantly associated with asthma in Caucasians, i.e. abnormal activity of the ecNOS gene predisposes Caucasians to asthma.

For African-Americans with cataracts due to HTN the odds ratio for the A allele was 2.4 (95% CI, 0.7-7.6). Data were not sufficient to generate genotypic odds ratios of 1.5 or greater. These data further suggest that the ecNOS gene is significantly associated with cataracts due to HTN in African-Americans, i.e. abnormal activity of the ecNOS gene predisposes African-Americans to cataracts due to HTN.

For Caucasians with cataracts due to HTN the odds ratio for the A allele was 1.6 (95% CI, 0.7-3.6). Data were not sufficient to generate genotypic odds ratios of 1.5 or greater. These data further suggest that the ecNOS gene is significantly associated with cataracts due to HTN in Caucasians, i.e. abnormal activity of the ecNOS gene predisposes Caucasians to cataracts due to HTN.

For Caucasians with colon cancer the odds ratio for the A allele was 1.6 (95% CI, 0.7-3.6). Data were not sufficient to generate genotypic odds ratios of 1.5 or greater. These data further suggest that the ecNOS gene is significantly associated with colon cancer in Caucasians, i.e. abnormal activity of the ecNOS gene predisposes Caucasians to colon cancer.

For African-Americans with DJD, or osteoarthritis, the odds ratio for the A allele was 1.9 (95% CI, 0.6-5.4). Data were not sufficient to generate genotypic odds ratios of 1.5 or greater. These data further suggest that the ecNOS gene is significantly associated with DJD in African-Americans, i.e. abnormal activity of the ecNOS gene predisposes African-Americans to DJD.

For African-Americans with ESRD due to IDDM the odds ratio for the 2 allele was 2.4 (95% CI, 0.7-7.6). Data were not sufficient to generate genotypic odds ratios of 1.5 or greater. These data further suggest that the ecNOS gene is significantly associated with ESRD due to IDDM in African-Americans, i.e. abnormal activity of the ecNOS gene predisposes African-Americans to ESRD due to IDDM.

For Caucasians with ESRD due to FSGS the odds ratio for the A allele was 2.1 (95% CI, 0.9-4.9). Data were not sufficient to generate genotypic odds ratios of 1.5 or greater. These data further suggest that the ecNOS gene is significantly associated with ESRD due to FSGS in Caucasians, i.e. abnormal activity of the ecNOS gene predisposes Caucasians to ESRD due to FSGS.

For African-Americans with CVA due to NIDDM the odds ratio for the G allele was 2.7 (95% CI, 0.5-14.5), compared to African-Americans with NIDDM only. The odds ratio for the homozygote (G/G) was 1.2^{H} (95% CI, 0-143.2), while the odds ratio for the heterozygote (G/A) was 2.9 (95% CI, 0.5-16.7). These data suggest that the G allele acts in a co-dominant manner in this patient population. These data further suggest that the ecNOS gene is significantly associated with CVA due to NIDDM in African-Americans, i.e. abnormal activity of the ecNOS gene predisposes African-Americans to CVA due to NIDDM.

For Caucasians with CVA due to NIDDM the odds ratio for the G allele was 1.8 (95% CI, 0.7-4.3), compared to Caucasians with NIDDM only. The odds ratio for the homozygote (G/G) was 10.7^{H} (95% CI, 0.2-629), while the odds ratio for the heterozygote (G/A) was 1.0 (95% CI, 0.3-3.3). These data suggest that the G allele acts in a recessive manner in this patient population. These data further suggest that the ecNOS gene is significantly associated with CVA due to NIDDM in Caucasians, i.e. abnormal activity of the ecNOS gene predisposes Caucasians to CVA due to NIDDM.

For African-Americans with CVA due to HTN the odds ratio for the G allele was 2.4 (95% CI, 0.7-8.5), compared to African-Americans with hypertension only. The odds ratio for the homozygote (G/G) was 3.5^{H} (95% CI, 0-251.4), while the odds ratio for the heterozygote (G/A) was 2.1 (95% CI, 0.5-8.4). These data suggest that the G allele acts in a dominant manner in this patient population with a greater than additive effect of allele dosage [3.5>4.2=(2.1+2.1−1.0)] (Goldstein et al., Monogr. Natl. Cancer Inst., 26:49-54, 1999). These data further suggest that the ecNOS gene is significantly associated with CVA due to HTN in African-Americans, i.e. abnormal activity of the ecNOS gene predisposes African-Americans to CVA due to HTN.

For African-Americans with seizure disorder the odds ratio for the A allele was 1.7 (95% CI, 0.6-4.9). Data were not sufficient to generate genotypic odds ratios of 1.5 or greater. These data further suggest that the ecNOS gene is significantly associated with seizure disorder in African-Americans, i.e. abnormal activity of the ecNOS gene predisposes African-Americans to seizure disorder.

Analysis

According to commercially available software [GENOMATIX MatInspector Professional; URL: http://genomatix.gsf.de/cgi-bin/matinspector/matinspector.pl; Quandt et al., Nucleic Acids Res. 23: 4878-4884 (1995)], the G2458→A SNP is predicted to have the following potential effects on transcription of the ecNOS gene:
a. Disruption of NF-1 (nuclear factor 1) site (5′-AGATGGCACAGAACTACA-3′ (SEQ ID NO: 4) beginning at position +2543 on the (+) strand. This polymorphism results in replacement of the indicated G by an A. NF-1 sites occur relatively frequently in the genome: 4.11 occasions per 1000 base pairs of random vertebrate genomic sequence. Since NF-1 is a positive transcriptional regulator, disruption of its binding site is expected to result in a decreased rate of transcription of the ecNOS gene. If the rate of translation is tied to the level of messenger RNA, as is the case for many proteins, then less gene product (ecNOS enzyme) will be the result, ultimately leading to less nitric oxide (NO) produced in tissues such as endothelial cells.
b. Disruption of MYOD (myoblast determining factor) binding site, which consists of 5′-GCCATCTGAG-3′ (SEQ ID NO: 5), ending at position +2540 on the (−) strand. Thus, this polymorphism results in replacement of the indicated C by a T on the (−) strand, since T is complementary to the polymorphic base, A, at this position on the (+) strand. MYOD binding sites are somewhat less frequent than NF1 sites, occurring 0.96 times per 1000 base pairs of random genomic sequence. MYOD is increasingly recognized as a potent transcriptional activator of more tissues than merely those destined to become skeletal muscle, in which context it was originally discovered. Again, this association suggests an unexpected biochemical mechanism for diabetic or hypertensive renal failure, in, e.g., black women, who express a higher frequency of the A allele. MYOD may operate in endothelial cells. It is possible that ecNOS production by smooth muscle cells, which are known to express MYOD, is important in regulation of renal blood flow and apoptosis of down-stream cellular elements.
c. Disruption of LMO2COM (complex of Lmo2 bound to Tal-1, E2A protein) binding site, which consists of the sequence 5′-CCTCAGATGGCA-3′ (SEQ ID NO: 6), beginning at position +2539 on the (+) strand. This polymorphism results in the replacement of the indicated G with an A. LMO2COM binding sites occur with a frequency of 1.11 times per 1000 base pairs of random genomic sequence, which is relatively frequent. The E2A protein is an adenoviral “early” protein, for which no cellular homolog is yet known.
d. Disruption of TAL1ALPHAE47 (Tal-1alpha/E47 heterodimer) binding site, which consists of the sequence 5′-CCCCTCAGATGGCACA-3′ (SEQ ID NO: 7), beginning at position +2537 on the (+) strand. This polymorphism results in the replacement of the indicated G with an A. TAL1ALPHAE47 binding sites occur rather infrequently, at the rate of 0.14 times per 1000 base pairs of random genomic sequence. Association of disease with this site thus suggests a novel mechanism for ecNOS regulation in cells whose identity is not yet known, but which could be endothelial, smooth muscle, mesangial, or tubular epithelial cells, for example.
e. Disruption of TAL1BETAE47 (Tal-1beta/E47 heterodimer) binding site, which consists also of the sequence 5′-CCCCTCAGATGGCACA-3′ (SEQ ID NO: 7), beginning at position +2537 on the (+) strand. This polymorphism results in the replacement of the indicated G with an A. TAL1BETAE47 binding sites occur rather infrequently, at the rate of 0.11 times per 1000 base pairs of random genomic sequence. Association of disease with this site thus suggests a novel mechanism for ecNOS regulation in cells whose identity is not yet known, but which could include endothelial, smooth muscle, mesangial, or tubular epithelial cells, for example.

### Example 3

### C to T Transition at Position 2684 of Human ecNOS Promoter

TABLE 9
ALLELE FREQUENCIES FOR GROUP I DISEASES

| | C | T |
|---|---|---|
| CONTROL | | |
| Black men (n = 84 chromosomes) | 10 (12%) | 74 (88%) |
| Black women (n = 74 chromosomes) | 18 (24%) | 56 (76%) |
| White men (n = 76 chromosomes) | 29 (38%) | 47 (62%) |
| White women (n = 94 chromosomes) | 29 (31%) | 65 (69%) |
| DISEASE | | |
| BREAST CANCER | | |
| Black women (n = 40 chromosomes) | 7 (18%) | 33 (82%) |
| White women (n = 38 chromosomes) | 12 (32%) | 26 (68%) |
| LUNG CANCER | | |
| Black men (n = 40 chromosomes) | 21 (53%) | 19 (48%) |
| Black women (n = 32 chromosomes) | 6 (19%) | 26 (81%) |
| White men (n = 40 chromosomes) | 17 (43%) | 23 (58%) |
| White women (n = 22 chromosomes) | 8 (36%) | 14 (64%) |
| PROSTATE CANCER | | |
| Black men (n = 40 chromosomes) | 9 (23%) | 31 (77%) |
| White men (n = 38 chromosomes) | 17 (45%) | 21 (55%) |
| NIDDM | | |
| Black men (n = 4 chromosomes) | 1 (25%) | 3 (75%) |
| Black women (n = 6 chromosomes) | 3 (50%) | 3 (50%) |
| White men (n = 8 chromosomes) | 0 (0%) | 8 (100%) |
| White women (n = 18 chromosomes) | 14 (78%) | 4 (22%) |
| ESRD due to NIDDM | | |
| Black men (n = 12 chromosomes) | 1 (8%) | 11 (92%) |
| Black women (n = 16 chromosomes) | 2 (13%) | 14 (88%) |
| White men (n = 10 chromosomes) | 2 (20%) | 8 (80%) |
| White women (n = 8 chromosomes) | 2 (25%) | 6 (75%) |
| HYPERTENSION (HTN) | | |
| Black men (n = 24 chromosomes) | 3 (13%) | 21 (88%) |
| Black women (n = 24 chromosomes) | 2 (8%) | 22 (92%) |
| White men (n = 22 chromosomes) | 7 (32%) | 15 (68%) |
| White women (n = 20 chromosomes) | 8 (40%) | 12 (60%) |
| ESRD due to HTN | | |
| Black men (n = 20 chromosomes) | 4 (20%) | 16 (80%) |
| Black women (n = 18 chromosomes) | 0 (0%) | 18 (100%) |
| White men (n = 18 chromosomes) | 5 (28%) | 13 (72%) |
| White women (n = 18 chromosomes) | 3 (17%) | 15 (83%) |
| MYOCARDIAL INFARCTION | | |
| White women (n = 14 chromosomes) | 5 (36%) | 9 (64%) |

TABLE 10
ALLELE FREQUENCY FOR GROUP II DISEASES

| Disease | Race | CHROMOSOMES | N | T | N | C |
|---|---|---|---|---|---|---|
| Controls | African-American | 92 | 75 | 81.5% | 17 | 18.5% |
| | Caucasian | 92 | 59 | 64.1% | 33 | 35.9% |
| Colon cancer | African-American | 48 | 42 | 87.5% | 6 | 12.5% |
| | Caucasian | 46 | 33 | 71.7% | 13 | 28.3% |
| Hypertension | African-American | 48 | 43 | 89.6% | 5 | 10.4% |
| | Caucasian | 44 | 27 | 61.4% | 17 | 38.6% |
| ASPVD due to HTN | African-American | 52 | 50 | 96.2% | 2 | 3.8% |
| | Caucasian | 50 | 46 | 92.0% | 4 | 8.0% |
| CVA due to HTN | African-American | 48 | 42 | 87.5% | 6 | 12.5% |
| | Caucasian | 48 | 32 | 66.7% | 16 | 33.3% |
| Cataracts due to HTN | African-American | 48 | 42 | 87.5% | 6 | 12.5% |
| | Caucasian | 44 | 33 | 75.0% | 11 | 25.0% |
| HTN CM | African-American | 48 | 43 | 89.6% | 5 | 10.4% |
| MI due to HTN | African-American | 42 | 41 | 97.6% | 1 | 2.4% |
| | Caucasian | 46 | 39 | 84.8% | 7 | 15.2% |
| ASPVD due to NIDDM | African-American | 48 | 41 | 85.4% | 7 | 14.6% |
| | Caucasian | 48 | 36 | 75.0% | 12 | 25.0% |
| CVA due to NIDDM | African-American | 48 | 45 | 93.8% | 3 | 6.3% |
| Ischemic CM | African-American | 48 | 36 | 75.0% | 12 | 25.0% |
| Ischemic CM with NIDDM | African-American | 48 | 45 | 93.8% | 3 | 6.3% |
| MI due to NIDDM | African-American | 48 | 42 | 87.5% | 6 | 12.5% |
| Afib without valvular disease | African-American | 48 | 41 | 85.4% | 7 | 14.6% |
| | Caucasian | 48 | 29 | 60.4% | 19 | 39.6% |
| Alcohol abuse | African-American | 48 | 40 | 83.3% | 8 | 16.7% |
| | Caucasian | 48 | 41 | 85.4% | 7 | 14.6% |
| Anxiety | African-American | 48 | 39 | 81.3% | 9 | 18.8% |
| Asthma | African-American | 46 | 40 | 87.0% | 6 | 13.0% |
| | Caucasian | 48 | 26 | 54.2% | 22 | 45.8% |
| COPD | African-American | 46 | 34 | 73.9% | 12 | 26.1% |
| | Caucasian | 42 | 27 | 64.3% | 15 | 35.7% |
| Cholecystectomy | African-American | 48 | 42 | 87.5% | 6 | 12.5% |
| | Caucasian | 46 | 27 | 58.7% | 19 | 41.3% |
| DJD | African-American | 42 | 38 | 90.5% | 4 | 9.5% |
| ESRD and frequent de-clots | African-American | 46 | 38 | 82.6% | 8 | 17.4% |
| | Caucasian | 42 | 30 | 71.4% | 12 | 28.6% |
| ESRD due to FSGS | African-American | 46 | 39 | 84.8% | 7 | 15.2% |
| | Caucasian | 46 | 35 | 76.1% | 11 | 23.9% |
| ESRD due to IDDM | African-American | 48 | 45 | 93.8% | 3 | 6.3% |
| Seizure disorder | African-American | 48 | 44 | 91.7% | 4 | 8.3% |
| | Caucasian | 48 | 32 | 66.7% | 16 | 33.3% |

TABLE 11
GENOTYPE FREQUENCIES FOR GROUP I DISEASES

| | C/C | C/T | T/T |
|---|---|---|---|
| CONTROLS | | | |
| Black men (n = 42) | 0 (0%) | 10 (24%) | 32 (76%) |
| Black women (n = 37) | 2 (5%) | 14 (38%) | 21 (57%) |
| White men (n = 38) | 5 (13%) | 19 (50%) | 14 (37%) |
| White women (n-47) | 2 (4%) | 25 (53%) | 20 (43%) |
| DISEASE | | | |
| BREAST CANCER | | | |
| Black women (n = 20) | 0 (0%) | 7 (35%) | 13 (65%) |
| White women (n = 19) | 1 (5%) | 10 (53%) | 8 (42%) |
| LUNG CANCER | | | |
| Black men (n = 20) | 8 (40%) | 5 (25%) | 7 (35%) |
| Black women (n = 16) | 0 (0%) | 6 (38%) | 10 (63%) |
| White men (n = 20) | 2 (10%) | 13 (65%) | 5 (25%) |
| White women (n = 11) | 2 (18%) | 4 (36%) | 5 (45%) |
| PROSTATE CANCER | | | |
| Black men (n = 20) | 0 (0%) | 9 (45%) | 11 (55%) |
| White men (n = 19) | 2 (11%) | 13 (68%) | 4 (21%) |
| NIDDM | | | |
| Black men (n = 2) | 0 (0%) | 1 (50%) | 1 (50%) |
| Black women (n = 3) | 1 (33%) | 1 (33%) | 1 (33%) |
| White men (n = 4) | 0 (0%) | 0 (0%) | 4(100%) |
| White women (n = 9) | 6 (67%) | 2 (22%) | 1 (11%) |
| ESRD due to NIDDM | | | |
| Black men (n = 6) | 0 (0%) | 1 (17%) | 5 (83%) |
| Black women (n = 8) | 0 (0%) | 2 (25%) | 6 (75%) |
| White men (n = 5) | 0 (0%) | 2 (40%) | 3 (60%) |
| White women (n = 4) | 0 (0%) | 2 (50%) | 2 (50%) |
| HYPERTENSION (HTN) | | | |
| Black men (n = 12) | 0 (0%) | 3 (25%) | 9 (75%) |
| Black women (n = 14) | 0 (0%) | 2 (17%) | 12 (83%) |
| White men (n = 11) | 1 (9%) | 5 (45%) | 5 (45%) |
| White women (n = 10) | 1 (10%) | 6 (60%) | 3 (30%) |
| ESRD due to HTN | | | |
| Black men (n = 10) | 1 (10%) | 2 (20%) | 7 (70%) |
| Black women (n = 9) | 0 (0%) | 0 (0%) | 9(100%) |
| White men (n = 9) | 0 (0%) | 5 (56%) | 4 (44%) |
| White women (n = 9) | 0 (0%) | 3 (33%) | 6 (67%) |
| MYOCARDIAL INFARCTION | | | |
| White women (n = 7) | 0 (0%) | 5 (71%) | 2 (29%) |

TABLE 12
GENOTYPE FREQUENCIES FOR GROUP II DISEASES

| Disease | Race | People | N | T/T | N | T/C | N | C/C |
|---|---|---|---|---|---|---|---|---|
| Controls | African-American | 46 | 31 | 67.4% | 13 | 28.3% | 2 | 4.3% |
| | Caucasian | 46 | 18 | 39.1% | 23 | 50.0% | 5 | 10.9% |
| Colon cancer | African-American | 24 | 18 | 75.0% | 6 | 25.0% | 0 | 0.0% |
| | Caucasian | 23 | 12 | 52.2% | 9 | 39.1% | 2 | 8.7% |
| Hypertension | African-American | 24 | 19 | 79.2% | 5 | 20.8% | 0 | 0.0% |
| | Caucasian | 22 | 8 | 36.4% | 11 | 50.0% | 3 | 13.6% |
| ASPVD due to HTN | African-American | 26 | 24 | 92.3% | 2 | 7.7% | 0 | 0.0% |
| | Caucasian | 25 | 21 | 84.0% | 4 | 16.0% | 0 | 0.0% |
| CVA due to HTN | African-American | 24 | 18 | 75.0% | 6 | 25.0% | 0 | 0.0% |
| | Caucasian | 24 | 12 | 50.0% | 8 | 33.3% | 4 | 16.7% |
| Cataracts due to HTN | African-American | 24 | 18 | 75.0% | 6 | 25.0% | 0 | 0.0% |
| | Caucasian | 22 | 11 | 50.0% | 11 | 50.0% | 0 | 0.0% |
| HTN CM | African-American | 24 | 19 | 79.2% | 5 | 20.8% | 0 | 0.0% |
| ASPVD due to NIDDM | African-American | 24 | 17 | 70.8% | 7 | 29.2% | 0 | 0.0% |
| | Caucasian | 24 | 13 | 54.2% | 10 | 41.7% | 1 | 4.2% |
| CVA due to NIDDM | African-American | 24 | 21 | 87.5% | 3 | 12.5% | 0 | 0.0% |
| Ischemic CM | African-American | 24 | 16 | 66.7% | 4 | 16.7% | 4 | 16.7% |
| Ischemic CM with NIDDM | African-American | 24 | 21 | 87.5% | 3 | 12.5% | 0 | 0.0% |
| Afib without valvular disease | African-American | 24 | 19 | 79.2% | 3 | 12.5% | 2 | 8.3% |
| | Caucasian | 24 | 9 | 37.5% | 11 | 45.8% | 4 | 16.7% |
| Alcohol abuse | African-American | 24 | 16 | 66.7% | 8 | 33.3% | 0 | 0.0% |
| | Caucasian | 24 | 17 | 70.8% | 7 | 29.2% | 0 | 0.0% |
| Anxiety | African-American | 24 | 16 | 66.7% | 7 | 29.2% | 1 | 4.2% |
| Asthma | African-American | 23 | 17 | 73.9% | 6 | 26.1% | 0 | 0.0% |
| | Caucasian | 24 | 8 | 33.3% | 10 | 41.7% | 6 | 25.0% |
| COPD | African-American | 23 | 13 | 56.5% | 8 | 34.8% | 2 | 8.7% |
| | Caucasian | 21 | 9 | 42.9% | 9 | 42.9% | 3 | 14.3% |
| Cholecystectomy | African-American | 24 | 18 | 75.0% | 6 | 25.0% | 0 | 0.0% |
| | Caucasian | 23 | 8 | 34.8% | 11 | 47.8% | 4 | 17.4% |
| DJD | African-American | 21 | 17 | 81.0% | 4 | 19.0% | 0 | 0.0% |
| ESRD and frequent de-clots | African-American | 23 | 17 | 73.9% | 4 | 17.4% | 2 | 8.7% |
| | Caucasian | 21 | 11 | 52.4% | 8 | 38.1% | 2 | 9.5% |
| ESRD due to FSGS | African-American | 23 | 16 | 69.6% | 7 | 30.4% | 0 | 0.0% |
| | Caucasian | 23 | 12 | 52.2% | 11 | 47.8% | 0 | 0.0% |
| ESRD due to IDDM | African-American | 24 | 21 | 87.5% | 3 | 12.5% | 0 | 0.0% |
| Seizure disorder | African-American | 24 | 20 | 83.3% | 4 | 16.7% | 0 | 0.0% |
| | Caucasian | 24 | 10 | 41.7% | 12 | 50.0% | 2 | 8.3% |

Allele-Specific Odds Ratios

The susceptibility allele is indicated, as well as the odds ratio (OR). Haldane's correction was used if the denominator was zero. If the odds ratio (OR) was ≧1.5, the 95% confidence interval (C.I.) is also given. An odds ratio of 1.5 was chosen as the threshold of significance based on the recommendation of Austin et al. in Epidemiol. Rev., 16:65-76, (1994). “[E]pidemiology in general and case-control studies in particular are not well suited for detecting weak associations (odds ratios <1.5).” Id. at 66. Odds ratios of 1.5 or higher are high-lighted below.

TABLE 13
ALLELE-SPECIFIC ODDS RATIOS FOR GROUP I DISEASES

| | SUSCEPTIBILITY | | |
|---|---|---|---|
| DISEASE | ALLELE | OR | 95% C.I. |
| Breast Cancer | | | |
| Black women | T | 1.5 | 0.6-4.0 |
| White women | C | 1.0 | |
| Lung Cancer | | | |
| Black men | C | 8.2 | 3.3-20 |
| Black women | T | 1.4 | |
| White men | T | 0.8 | |
| White women | C | 1.3 | |
| Prostate Cancer | | | |
| Black men | C | 2.1 | 0.8-5.8 |
| White men | C | 0.8 | |
| NIDDM | | | |
| Black men | C | 2.5 | 0.2-26 |
| Black women | C | 3.1 | 0.6-17 |
| White men | T | 10.6 | 1.4-81 |
| White women | C | 7.8 | 2.4-26 |
| ESRD due to NIDDM* | | | |
| Black men | T | 3.7 | 0.2-78 |
| Black women | T | 7.0 | 0.8-62 |
| White men | C | 5.0 | 0.5-47 |
| White women | T | 10.5 | 1.5-74 |
| Hypertension (HTN) | | | |
| Black men | C | 1.1 | |
| Black women | T | 3.5 | 0.8-17 |
| White men | T | 1.3 | |
| White women | C | 1.5 | 0.6-40 |
| ESRD due to HTN*¹ | | | |
| Black men | C | 1.8 | 0.3-9.0 |
| Black women | T | 4.1 | 0.5-37 |
| White men | T | 1.2 | |
| White women | T | 2.3 | 0.5-11 |
| Myocardial Infarction | | | |
| White women | C | 1.2 | |

*Compared to group with NIDDM alone.
*¹Compared to group with HTN alone.

TABLE 14
ALLELE-SPECIFIC ODDS RATIOS FOR GROUP II DISEASES

| | | | | Lower | Upper | |
|---|---|---|---|---|---|---|
| | | | | Limit | Limit | |
| | | Risk | Odds | 95% | 95% | |
| Disease | Race | Allele | Ratio | CI | CI | Haldane |
| Colon cancer | African-American | T | 1.6 | 0.6 | 4.3 | |
| | Caucasian | T | 1.4 | 0.7 | 3.1 | |
| ASPVD due to HTN* | African-American | T | 2.9 | 0.5 | 15.8 | |
| | Caucasian | T | 7.2 | 2.2 | 23.8 | |
| CVA due to HTN* | African-American | C | 1.2 | 0.3 | 4.3 | |
| | Caucasian | T | 1.3 | 0.5 | 3.0 | |
| Cataracts due to HTN* | African-American | T | 1.6 | 0.6 | 4.3 | |
| | Caucasian | T | 1.7 | 0.8 | 3.8 | |
| Afib without valvular disease | African-American | T | 1.3 | 0.5 | 3.5 | |
| | Caucasian | C | 1.2 | 0.6 | 2.4 | |
| Alcohol abuse | African-American | T | 1.1 | 0.4 | 2.9 | |
| | Caucasian | T | 3.3 | 1.3 | 8.1 | |
| Anxiety | African-American | C | 1.0 | 0.4 | 2.5 | |
| Asthma | African-American | T | 1.5 | 0.6 | 4.1 | |
| | Caucasian | C | 1.5 | 0.7 | 3.1 | |
| COPD | African-American | C | 1.6 | 0.7 | 3.6 | |
| | Caucasian | T | 1.0 | 0.5 | 2.2 | |
| Cholecystectomy | African-American | T | 1.6 | 0.6 | 4.3 | |
| | Caucasian | C | 1.3 | 0.6 | 2.6 | |
| DJD | African-American | T | 2.2 | 0.7 | 6.8 | |
| ESRD and frequent de-clots | African-American | T | 1.1 | 0.4 | 2.7 | |
| | Caucasian | T | 1.4 | 0.6 | 3.1 | |
| ESRD due to FSGS | African-American | T | 1.3 | 0.5 | 3.3 | |
| | Caucasian | T | 1.8 | 0.8 | 4.0 | |
| ESRD due to IDDM | African-American | T | 3.4 | 0.9 | 12.3 | |
| Seizure disorder | African-American | T | 2.5 | 0.8 | 7.9 | |
| | Caucasian | T | 1.1 | 0.5 | 2.3 | |

Genotype-Specific Odds Ratios

The susceptibility allele (S) is indicated, and the alternative allele at this locus is defined as the protective allele (P). Also presented is the odds ratio (OR) for the SS and SP genotypes. The odds ratio for the PP genotype is 1 by definition, since it is the reference group, and is not presented in the table below. For odds ratios ≧1.5, the asymptotic 95% confidence interval (C.I.) is also given, in parentheses. An odds ratio of 1.5 was chosen as the threshold of significance based on the recommendation of Austin et al., in Epidemiol. Rev., 16:65-76 (1994). “[E]pidemiology in general and case-control studies in particular are not well suited for detecting weak associations (odds ratios <1.5).” Id. at 66.

Odds ratios of 1.5 or higher are high-lighted below. Haldane's correction was used when the denominator was zero.

TABLE 15
GENOTYPE-SPECIFIC ODDS RATIOS FOR GROUP I DISEASES

| | SUSCEPTIBILITY | | |
|---|---|---|---|
| DISEASE | ALLELE | OR(SS) | OR(SP) |
| Breast Cancer | | | |
| Black women | T | 3.1 (0.3-28) | 2.6 (0.3-24) |
| Lung Cancer | | | |
| Black men | C | 74 (9.1-598) | 2.3 (0.9-5.7) |
| Prostate Cancer | | | |
| Black men | C | 2.8 (0.2-47) | 2.6 (1.2-5.6) |
| NIDDM | | | |
| Black men | C | 22 (1.1-437) | 3.1 (0.6-17) |
| Black women | C | 11 (0.5-240) | 1.5 (0.1-26) |
| White men | T | 3.4 (0.4-30) | 0.3 |
| White women | C | 60 (4.6-782) | 1.6 (0.1-19) |
| ESRD due | | | |
| to NIDDM* | | | |
| Black men | T | 3.7 (0.2-78) | 1.0 |
| Black women | T | 13 (1.0-173) | 5.0 (0.3-73) |
| White men | C | 1.3 | 6.4 (0.6-68) |
| White women | T | 22 (1.8-261) | 13 (1.2-141) |
| Hypertension | | | |
| (HTN) | | | |
| Black women | T | 2.9 (0.3-26) | 0.9 |
| White women | C | 3.3 (0.2-49) | 1.6 (0.4-7.2) |
| ESRD due | | | |
| to HTN*¹ | | | |
| Black men | C | 3.8 (0.4-40) | 0.9 |
| Black women | T | 0.8 | 0.2 |
| White women | T | 5.6 (0.5-64) | 1.6 (0.1-19) |

*Compared to group with NIDDM alone.
*¹Compared to group with HTN alone.

TABLE 16
GENOTYPE-SPECIFIC ODDS RATIOS FOR GROUP II DISEASES

| | | | | Lower | Upper | |
|---|---|---|---|---|---|---|
| | | | | Limit | Limit | |
| | | Risk | Odds | 95% | 95% | |
| Disease | Race | Allele | Ratio | CI | CI | Haldane |
| Colon cancer | African-American | T | 1.6 | 0.6 | 4.3 | |
| | Caucasian | T | 1.4 | 0.7 | 3.1 | |
| ASPVD due to HTN* | African-American | T | 2.9 | 0.5 | 15.8 | |
| | Caucasian | T | 7.2 | 2.2 | 23.8 | |
| CVA due to HTN* | African-American | C | 1.2 | 0.3 | 4.3 | |
| | Caucasian | T | 1.3 | 0.5 | 3.0 | |
| Cataracts due to HTN* | African-American | T | 1.6 | 0.6 | 4.3 | |
| | Caucasian | T | 1.7 | 0.8 | 3.8 | |
| Afib without valvular disease | African-American | T | 1.3 | 0.5 | 3.5 | |
| | Caucasian | C | 1.2 | 0.6 | 2.4 | |
| Alcohol abuse | African-American | T | 1.1 | 0.4 | 2.9 | |
| | Caucasian | T | 3.3 | 1.3 | 8.1 | |
| Anxiety | African-American | C | 1.0 | 0.4 | 2.5 | |
| Asthma | African-American | T | 1.5 | 0.6 | 4.1 | |
| | Caucasian | C | 1.5 | 0.7 | 3.1 | |
| COPD | African-American | C | 1.6 | 0.7 | 3.6 | |
| | Caucasian | T | 1.0 | 0.5 | 2.2 | |
| Cholecystectomy | African-American | T | 1.6 | 0.6 | 4.3 | |
| | Caucasian | C | 1.3 | 0.6 | 2.6 | |
| DJD | African-American | T | 2.2 | 0.7 | 6.8 | |
| ESRD and frequent de-clots | African-American | T | 1.1 | 0.4 | 2.7 | |
| | Caucasian | T | 1.4 | 0.6 | 3.1 | |
| ESRD due to FSGS | African-American | T | 1.3 | 0.5 | 3.3 | |
| | Caucasian | T | 1.8 | 0.8 | 4.0 | |
| ESRD due to IDDM | African-American | T | 3.4 | 0.9 | 12.3 | |
| Seizure disorder | African-American | T | 2.5 | 0.8 | 7.9 | |
| | Caucasian | T | 1.1 | 0.5 | 2.3 | |

*Compared to group with Hypertension alone.

PCR and sequencing were conducted as in Example 1. The primers were the same as in Example 1. The control samples agree with Hardy-Weinberg equilibrium, as follows:

In the control group for the Group I Diseases, a frequency of 0.12 for the C allele (“p”) and 0.88 for the T allele (“q”) among black male control individuals predicts genotype frequencies of 1% C/C, 22% C/T, and 77% T/T at Hardy-Weinberg equilibrium (p²+2pq+q²=1). The observed genotype frequencies were 0% C/C, 24% C/T, and 76% T/T, in excellent agreement with those predicted for Hardy-Weinberg equilibrium.

A frequency of 0.24 for the C allele (“p”) and 0.76 for the T allele (“q”) among black female control individuals predicts genotype frequencies of 6% C/C, 36% C/T, and 58% T/T at Hardy-Weinberg equilibrium (p²+2pq+q²=1). The observed genotype frequencies were 5% C/C, 38% C/T, and 57% T/T, in excellent agreement with those predicted for Hardy-Weinberg equilibrium.

A frequency of 0.38 for the C allele (“p”) and 0.62 for the T allele (“q”) among white male control individuals predicts genotype frequencies of 14% C/C, 48% C/T, and 38% T/T at Hardy-Weinberg equilibrium (p²+2pq+q²=1). The observed genotype frequencies were 13% C/C, 50% C/T, and 37% T/T, in excellent agreement with those predicted for Hardy-Weinberg equilibrium.

A frequency of 0.31 for the C allele (“p”) and 0.69 for the T allele (“q”) among white female control individuals predicts genotype frequencies of 10% C/C, 42% C/T, and 48% T/T at Hardy-Weinberg equilibrium (p²+2pq+q²=1). The observed genotype frequencies were 4% C/C, 53% C/T, and 43% T/T, in fair agreement with those predicted for Hardy-Weinberg equilibrium.

In the control group for the Group II Diseases, a frequency of 0.18 for the C allele (“p”) and 0.82 for the T allele (“q”) among African-American control individuals predicts genotype frequencies 3.2% C/C, 29.5% C/T, and 67.2% T/T at Hardy-Weinberg equilibrium (p²+2pq+q²=1). The observed genotype frequencies were 4.3% C/C, 28.3% C/T, and 67.4% T/T, in almost perfect agreement with those predicted for Hardy-Weinberg equilibrium.

A frequency of 0.36 for the C allele (“p”) and 0.64 for the T allele (“q”) among Caucasian control individuals predicts genotype frequencies of 12.9% C/C, 46.1% C/T, and 41.0% T/T at Hardy-Weinberg equilibrium (p²+2pq+q²=1). The observed genotype frequencies were 10.9% C/C, 50.0% C/T, and 39.1 % T/T, in excellent agreement with those predicted for Hardy-Weinberg equilibrium.

Results

Using an allele-specific odds ratio of 1.5 or greater as a practical level of significance, the following observations can be made.

Among black women with breast cancer, the odds ratio for the T allele at this locus was 1.5 (95% CI, 0.6-4.0). The odds ratio for the TC heterozygote was 2.6 (95% CI, 0.3-24), and 3.1 (95% CI, 0.3-28) for the TT homozygote. The genotype-specific odds ratios suggest that the T allele behaves as a dominant susceptibility allele.

For black men with lung cancer, the odds ratio for the C allele at this locus was 8.2 (95% CI, 3.3-20). The odds ratio for the CT heterozygote was 2.3 (95% CI, 0.9-5.7), and a remarkable 74 (95% CI, 9.1-598) for the CC homozygote. The genotype-specific odds ratios suggest that the T allele behaves as a dominant susceptibility allele, since the heterozygote (with one allele copy) has an odds ratio of 2.3. However, there is a pronounced (more than multiplicative) effect of gene dosage, since the homozygote with two copies of the C allele displayed a more than 30-fold larger odds ratio.

For black men with prostate cancer, the odds ratio for the C allele at this locus was 2.1 (95% CI, 0.8-5.8). The odds ratio for the heterozygote (2.6, 95% CI, 1.2-5.6) was essentially the same as for the CC homozygote (2.8, 95% CI, 0.2-47), suggesting that the C allele behaves in a dominant fashion.

For black men with NIDDM, the odds ratio for the C allele at this locus was 2.5 (95% CI, 0.2-26). The odds ratio for the heterozygote was 3.1 (95% CI, 0.6-17), and for the CC homozygote was a remarkable 22 (95% CI, 1.1-437). The genotype-specific odds ratios suggest that the C allele behaves as a dominant susceptibility allele, since the heterozygote (with one allele copy) had an odds ratio of 3.1. However, there is a pronounced effect of gene dosage, since the homozygote with two copies of the C allele displayed a more than 7-fold larger odds ratio than the heterozygote.

For black women with NIDDM, the odds ratio for the C allele at this locus was 3.1 (95% CI, 0.6-17). The odds ratio for the heterozygote was 1.5 (95% CI, 0.1-26), and for the CC homozygote was a remarkable 11 (95% CI, 0.5-240). The genotype-specific odds ratios suggest that the C allele behaves as a dominant susceptibility allele, since the heterozygote (with one allele copy) had an odds ratio of 1.5. However, there is a pronounced (more than multiplicative) effect of gene dosage, since the homozygote with two copies of the C allele displayed a more than 7-fold larger odds ratio than the heterozygote.

For white men with NIDDM, the odds ratio for the T allele at this locus was 10.6 (95% CI, 1.4-81). The odds ratio for the heterozygote was actually less than one (0.3), but for the TT homozygote was 3.4 (95% CI, 0.4-30). The genotype-specific odds ratios suggest that the T allele behaves as a recessive susceptibility allele.

For white women with NIDDM, the odds ratio for the C allele at this locus was 7.8 (95% CI, 2.4-26). The odds ratio for the heterozygote was 1.6 (95% CI, 0.1-19), and for the CC homozygote was a remarkable 60 (95% CI, 4.6-782). The genotype-specific odds ratios suggest that the C allele behaves as a dominant susceptibility allele, since the heterozygote (with one allele copy) had an odds ratio of 1.6. However, there is a pronounced (more than multiplicative) effect of gene dosage, since the homozygote with two copies of the C allele displayed a more than 37-fold larger odds ratio than the heterozygote.

For black men with ESRD due to NIDDM, the odds ratio for the T allele at this locus was 3.7 (95% CI, 0.2-78), compared with black men with NIDDM but no renal disease. The odds ratio for the heterozygote was 1.0, but for the TT homozygote was 3.7 (95% CI, 0.2-78). The genotype-specific odds ratios suggest that the T allele behaves as a recessive susceptibility allele.

For black women with ESRD due to NIDDM, the odds ratio for the T allele at this locus was 7.0 (95% CI, 0.8-62), compared with black women with NIDDM but no renal disease. The odds ratio for the heterozygote was 5.0 (95% CI, 0.3-73), and for the TT homozygote was 13 (95% CI, 1.0-173). The genotype-specific odds ratios suggest that the T allele behaves as a dominant susceptibility allele. However, there is a pronounced (more than additive) effect of gene dosage, since the homozygote with two copies of the C allele displayed a more than two-fold larger odds ratio than the heterozygote.

For white men with ESRD due to NIDDM, the odds ratio for the C allele at this locus was 5.0 (95% CI, 0.547) vs. white men with NIDDM but no renal disease. Inspection of the genotype-specific odds ratios suggests that the C allele is codominant, since the heterozygote had a much higher odds ratio (6.4, 95% CI 0.6-68) than the CC homozygote (1.3) or the reference TT genotype (odds ratio 1, by definition).

For white women with ESRD due to NIDDM, the odds ratio for the T allele at this locus was 10.5 (95% CI, 1.5-74) vs. white women with NIDDM but no renal disease. The odds ratio for the heterozygote was 13 (95% CI, 1.2-141), and the TT homozygote was 22 (95% CI, 1.8-261). The genotype-specific odds ratios suggest that the T allele behaves as a dominant susceptibility allele. However, there is a pronounced (approximately additive) effect of gene dosage, since the homozygote with two copies of the T allele displayed a roughly two-fold larger odds ratio than the heterozygote.

For black women with hypertension, the odds ratio for the T allele at this locus was 3.5 (95% CI, 0.8-17). The odds ratio for the heterozygote was 0.9, but for the TT homozygote was 2.9 (95% CI, 0.3-26). The genotype-specific odds ratios suggest that the T allele behaves as a recessive susceptibility allele.

For white women with hypertension, the odds ratio for the C allele at this locus was 1.5 (95% CI, 0.6-40). The odds ratio for the heterozygote was 1.6 (95% CI, 0.4-7.2), and for the CC homozygote was 3.3 (95% CI, 0.2-49). The genotype-specific odds ratios suggest that the C allele behaves in a dominant fashion, with a strictly additive effect of allele dosage, since 1.6+1.6˜3.3.

For black men with ESRD due to hypertension (HTN), the odds ratio for the C allele at this locus was 1.8 (95% CI, 0.3-9.0) relative to black men with HTN but no renal failure. The odds ratio for the heterozygote was 0.9, but for the CC homozygote was 3.8 (95% CI, 0.4-40). The genotype-specific odds ratios suggest that the C allele behaves in a recessive fashion.

For black women with ESRD due to HTN, the odds ratio for the T allele was 4.1 (95% CI, 0.5-37) relative to black women with HTN alone. The genotype-specific odds ratios were found to be unhelpful, so no inference can be drawn about whether the T allele behaves in a dominant, recessive, or codominant fashion.

For white women with ESRD due to HTN, the odds ratio for the T allele was 2.3 (95% CI, 0.5-11) relative to white women with HTN alone. The odds ratio for the heterozygote was 1.6 (95% CI, 0.1-19), and for the TT homozygote was 5.6 (95% CI, 0.5-64). The genotype-specific odds ratios suggest that the C allele behaves in a dominant fashion, with a more than multiplicative effect of allele dosage, since 5.6/(1.6)²=5.6/3.56=1.6>1.

For Caucasians with alcohol abuse the odds ratio for the T allele was 3.3 (95% CI, 1.3-8.1). The odds ratio for the homozygote (T/T) was 10.4^{H} (95% CI, 0.6-186.1), while the odds ratio for the heterozygote (T/C) was 3.5^{H} (95% CI, 0.2-71.2). These data suggest that the T allele acts in a dominant manner in this patient population with a greater than additive effect of allele dosage [10.4>7=(3.5+3.5−1.0)]. (Goldstein et al., Monogr. Natl. Cancer Inst., 26:49-54, 1999). These data further suggest that the ecNOS gene is significantly associated with alcohol abuse in Caucasians, i.e. abnormal activity of the ecNOS gene predisposes Caucasians to alcohol abuse.

For African-Americans with ASPVD due to HTN the odds ratio for the T allele was 2.9 (95% CI, 0.5-15.8), compared to African-Americans with hypertension only. Data were not sufficient to generate genotypic odds ratios of 1.5 or greater. These data further suggest that the ecNOS gene is significantly associated with ASPVD due to HTN in African-Americans, i.e. abnormal activity of the ecNOS gene predisposes African-Americans to ASPVD due to HTN.

For Caucasians with ASPVD due to HTN the odds ratio for the T allele was 7.2 (95% CI, 2.2-23.8), compared to Caucasians with hypertension only. The odds ratio for the homozygote (T/T) was 17.7^{H} (95% CI, 0.9-341.9), while the odds ratio for the heterozygote (T/C) was 2.7^{H} (95% CI, 0.1-64.4). These data suggest that the T allele acts in a dominant manner in this patient population with a greater than multiplicative effect of allele dosage [17.7>7.29=(2.7)(2.7)]. These data further suggest that the ecNOS gene is significantly associated with ASPVD due to HTN in Caucasians, i.e. abnormal activity of the ecNOS gene predisposes Caucasians to ASPVD due to HTN.

For African-Americans with cataracts due to HTN the odds ratio for the T allele was 1.6 (95% CI, 0.6-4.3). The odds ratio for the homozygote (T/T) was 2.9^{H} (95% CI, 0.2-50.9), while the odds ratio for the heterozygote (T/C) was 2.4^{H} (95% CI, 0.1-57.7). These data suggest that the T allele acts in a dominant manner in this patient population with a less than additive effect of allele dosage [2.9<4.8=(2.4+2.4−1.0)]. (Goldstein et al., Monogr. Natl. Cancer Inst., 26:49-54, 1999). These data further suggest that the ecNOS gene is significantly associated with cataracts due to HTN in African-Americans, i.e. abnormal activity of the ecNOS gene predisposes African-Americans to cataracts due to HTN.

For Caucasians with cataracts due to HTN the odds ratio for the T allele was 1.7 (95% CI, 0.8-3.8). The odds ratio for the homozygote (T/T) was 6.8^{H} (95% CI, 0.4-124.8), while the odds ratio for the heterozygote (T/C) was 5.4^{H} (95 % CI, 0.3-106). These data suggest that the T allele acts in a dominant manner in this patient population with a less than additive effect of allele dosage [6.8<10.8=(5.4+5.4−1.0)]. (Goldstein et al., Monogr. Nat. Cancer Inst., 26:49-54, 1999). These data further suggest that the ecNOS gene is significantly associated with cataracts due to HTN in Caucasians, i.e. abnormal activity of the ecNOS gene predisposes Caucasians to cataracts due to HTN.

For African-Americans with cholecystectomy the odds ratio for the T allele was 1.6 (95% CI, 0.6-4.3). The odds ratio for the homozygote (T/T) was 2.9^{H} (95% CI, 0.2-50.9), while the odds ratio for the heterozygote (T/C) was 2.4^{H} (95% CI, 0.1-57.7). These data suggest that the T allele acts in a dominant manner in this patient population with a less than additive effect of allele dosage [2.9<4.8=(2.4+2.4−1.0)]. (Goldstein et al., Monogr. Natl. Cancer Inst., 26:49-54, 1999). These data further suggest that the ecNOS gene is significantly associated with cholecystectomy in African-Americans, i.e. abnormal activity of the ecNOS gene predisposes African-Americans to cholecystectomy.

For African-Americans with colon cancer the odds ratio for the T allele was 1.6 (95% CI, 0.6-4.3). The odds ratio for the homozygote (T/T) was 2.9^{H} (95% CI, 0.2-50.9), while the odds ratio for the heterozygote (T/C) was 2.4^{H} (95% CI, 0.1-57.7). These data suggest that the T allele acts in a dominant manner in this patient population with a less than additive effect of allele dosage [2.9<4.8=(2.4+2.4−1.0)]. (Goldstein et al., Monogr. Natl. Cancer Inst., 26:49-54, 1999). These data further suggest that the ecNOS gene is significantly associated with colon cancer in African-Americans, i.e. abnormal activity of the ecNOS gene predisposes African-Americans to colon cancer.

For African-Americans with COPD the odds ratio for the C allele was 1.6 (95% CI, 0.7-3.6). Data were not sufficient to generate genotypic odds ratios of 1.5 or greater. These data further suggest that the ecNOS gene is significantly associated with COPD in African-Americans, i.e. abnormal activity of the ecNOS gene predisposes African-Americans to COPD.

For African-Americans with DJD (osteoarthritis) the odds ratio for the T allele was 2.2 (95% CI, 0.7-6.8). The odds ratio for the homozygote (T/T) was 2.8^{H} (95% CI, 0.2-48.2), while the odds ratio for the heterozygote (T/C) was 1.7^{H} (95% CI, 0.1-41.6). These data suggest that the T allele acts in a dominant manner in this patient population with a greater than additive effect of allele dosage [2.8>3.4=(1.7+1.7−1.0)]. (Goldstein et al., Monogr. Natl. Cancer Inst., 26:49-54, 1999). These data further suggest that the ecNOS gene is significantly associated with DJD (osteoarthritis) in African-Americans, i.e. abnormal activity of the ecNOS gene predisposes African-Americans to DJD (osteoarthritis).

For African-Americans with ESRD due to IDDM the odds ratio for the T allele was 3.4 (95% CI, 0.9-12.3). The odds ratio for the homozygote (T/T) was 3.4^{H} (95% CI, 0.2-58.8), while the odds ratio for the heterozygote (T/C) was 1.3^{H} (95% CI, 0-33.6). These data suggest that the T allele acts in a recessive manner in this patient population. These data further suggest that the ecNOS gene is significantly associated with ESRD due to IDDM in African-Americans, i.e. abnormal activity of the ecNOS gene predisposes African-Americans to ESRD due to IDDM.

For Caucasians with ESRD due to FSGS the odds ratio for the T allele was 1.8 (95% CI, 0.8-4). The odds ratio for the homozygote (T/T) was 7.4^{H} (95% CI, 0.4-135), while the odds ratio for the heterozygote (T/C) was 5.4^{H} (95% CI, 0.3-106). These data suggest that the T allele acts in a dominant manner in this patient population with a less than additive effect of allele dosage [7.4<10.8=(5.4+5.4−1.0)]. (Goldstein et al., Monogr. Natl. Cancer Inst., 26:49-54, 1999). These data further suggest that the ecNOS gene is significantly associated with ESRD due to FSGS in Caucasians, i.e. abnormal activity of the ecNOS gene predisposes Caucasians to ESRD due to FSGS.

For African-Americans with hypertension only the odds ratio for the T allele was 1.9 (95% CI, 0.7-5.7). The odds ratio for the homozygote (T/T) was 3.1^{H} (95% CI, 0.2-53.5), while the odds ratio for the heterozygote (T/C) was 2.0^{H} (95% CI, 0.1-49.7). These data suggest that the T allele acts in a dominant manner in this patient population with a greater than additive effect of allele dosage [3.1>4=(2+2−1.0)]. (Goldstein et al., Monogr. Natl. Cancer Inst., 26:49-54, 1999). These data further suggest that the ecNOS gene is significantly associated with hypertension only in African-Americans, i.e. abnormal activity of the ecNOS gene predisposes African-Americans to hypertension only.

For African-Americans with seizure disorder the odds ratio for the T allele was 2.5 (95% CI, 0.8-7.9). The odds ratio for the homozygote (T/T) was 3.3^{H} (95% CI, 0.2-56.2), while the odds ratio for the heterozygote (T/C) was 1.7^{H} (95% CI,0.1-41.6). These data suggest that the T allele acts in a dominant manner in this patient population. These data further suggest that the ecNOS gene is significantly associated with seizure disorder in African-Americans, i.e. abnormal activity of the ecNOS gene predisposes African-Americans to seizure disorder.

Analysis

According to commercially available software [GENOMATIX MatInspector Professional; URL:http://genomatix.gsf.de/cgi-bin/matinspector/matinspector.pl; Quandt et al., Nucleic Acids Res. 23: 4878-4884 (1995)], the C2684-→T SNP is predicted to have the following potential effects on transcription of the ecNOS gene:
a. Disruption of an NF1 (nuclear factor 1) binding site, which consists of the sequence 5′-CCCTGGCCGGCTGACCCT-3′(SEQ ID NO: 8), beginning at position +2677 on the (+) strand. This polymorphism replaces the indicated C with a T, which should result in a weaker binding site for NF1, a transcriptional activator of ecNOS. NF1 binding sites occur rather frequently, 4.11 times per 1000 base pairs of random genomic sequence. Since NF-1 is a positive transcriptional regulator, disruption of its binding site is expected to result in a decreased rate of transcription of the ecNOS gene. If the rate of translation is tied to the level of messenger RNA, as is the case for most proteins, then less gene product (ecNOS enzyme) will be the result, ultimately leading to less nitric oxide (NO) produced in tissues such as endothelial cells.
b. Disruption of an ER (estrogen receptor) binding site, which consists of the sequence 5′-CCCTGGCCGGCTGACCCT-3′(SEQ ID NO: 8), beginning at position +2677 on the (+) strand. This polymorphism replaces the indicated C with a T, which should result in a weaker binding site for the estrogen receptor, a transcriptional activator of ecNOS. ER binding sites occur moderately frequently, at the rate of 1.73 sites per 1000 base pairs of random genomic sequence. Since the estrogen receptor is a transcriptional activator, disruption of its binding site is expected to result in a decreased rate of transcription of the ecNOS gene. If the rate of translation is tied to the level of messenger RNA, as is the case for most proteins, then less gene product (ecNOS enzyme) will be the result, ultimately leading to less nitric oxide (NO) produced in tissues such as endothelial cells. In rodents, androgens have been shown to accelerate renal failure. Thus, it is intriguing that this polymorphism might interfere with the effect of estrogen, essentially tilting the balance towards androgens.
c. Disruption of a TCF11 (TCF11/KCR-F1/Nrf1 homodimer) binding site, which consists of the sequence 5′-GTCAGCCGGCCAG-3′(SEQ ID NO: 9), which ends at position +2679 on the (−) strand. This polymorphism replaces the C on the (+) strand by a T on the (+) strand. The complementary base on the (−) strand is thus changed from the wild type G, indicated in TCFL11's bindin site, above, to an A, complementary to the T of the polymorphism. The TCF11 binding site occurs rather frequently, at the rate of 4.63 times per 1000 base pairs of random genomic sequence. Involvement of the TCF11 homodimer in regulation of ecNOS has not previously been demonstrated.
d. Disruption of an AP4 (activator protein 4) binding site, which consists of the sequence 5′-GTCAGCCGGC-3′(SEQ ID NO: 10), which ends at position +2682 on the (−) strand. The C2684→T polymorphism replaces the C on the (+) strand by a T on the (+) strand. The complementary base on the (−) strand thus becomes A, rather than the wild type G as indicated immediately above. AP4 is a potent transcriptional activator. Its sites occur with only moderate frequency in genomic DNA: 0.96 times per 1000 base pairs in a random genomic sequence of vertebrates. Disruption of an AP4 site is predicted to lead to a decrease in transcription of the ecNOS gene, with a resultant decrease in tissue nitric oxide production.
e. Disruption of a VMAF (v-Maf) binding site, which consists of the sequence 5′-GCCGGCTGACCCTGCCTCA-3′(SEQ ID NO: 11), beginning at position +2682 on the (+) strand. Thus, the C2684→T polymorphism replaces the indicated C by a T. VMAF sites occur moderately frequently, i.e., 0.99 times per 1000 base pairs of random genomic sequence in vertebrates. At the moment, very little is known about the regulation of ecNOS by the cellular homolog of v-Maf.

Sim et al., Mol. Genet. Metab., 65: 562 (1998), reported a disruption of a MspI restriction site in the ecNOS gene. However, the specific MspI site reported in Sim et al., was not flirter identified by sequencing, and there are 11 MspI restriction sites predicted in the sequence we have examined (GenBank Accession Number AF032908).

### Example 4

### G to A Transition at Position 2701 of Human ecNOS Promoter

TABLE 17
ALLELE FREQUENCIES FOR GROUP I DISEASES

| | G | A |
|---|---|---|
| CONTROL | | |
| Black men (n = 6 chromosomes) | 6 (100%) | 0 (0%) |
| Black women (n = 2 chromosomes) | 1 (50%) | 1 (50%) |
| White men (n = 8 chromosomes) | 5 (63%) | 3 (38%) |
| White women (n = 14 chromosomes) | 9 (64%) | 5 (36%) |
| DISEASE | | |
| BREAST CANCER | | |
| Black women (n = 16 chromosomes) | 16 (100%) | 0 (0%) |
| White women (n = 14 chromosomes) | 13 (93%) | 1 (7%) |
| LUNG CANCER | | |
| Black men (n = 16 chromosomes) | 16 (100%) | 0 (0%) |
| Black women (n = 16 chromosomes) | 16 (100%) | 0 (0%) |
| White men (n = 16 chromosomes) | 16 (100%) | 0 (0%) |
| White women (n = 2 chromosomes) | 2 (100%) | 0 (0%) |
| PROSTATE CANCER | | |
| Black men (n = 16 chromosomes) | 16 (100%) | 0 (0%) |
| White men (n = 16 chromosomes) | 16 (100%) | 0 (0%) |
| NIDDM | | |
| Black men (n = 4 chromosomes) | 2 (50%) | 2 (50%) |
| Black women (n = 6 chromosomes) | 1 (17%) | 5 (83%) |
| White men (n = 8 chromosomes) | 2 (25%) | 6 (75%) |
| White women (n = 6 chromosomes) | 5 (83%) | 1 (17%) |
| ESRD due to NIDDM | | |
| Black men (n = 12 chromosomes) | 12 (100%) | 0 (0%) |
| Black women (n = 16 chromosomes) | 16 (100%) | 0 (0%) |
| White men (n = 10 chromosomes) | 10 (100%) | 0 (0%) |
| White women (n = 8 chromosomes) | 8 (100%) | 0 (0%) |
| MYOCARDIAL INFARCTION | | |
| White women (n = 14 chromosomes) | 14 (100%) | 0 (0%) |

TABLE 18
GENOTYPE FREQUENCIES FOR GROUP I DISEASES

| | G/G | G/A | A/A |
|---|---|---|---|
| CONTROLS | | | |
| Black men (n = 3) | 3 (100%) | 0 (0%) | 0 (0%) |
| Black women (n = 1) | 0 (0%) | 1 (100%) | 0 (0%) |
| White men (n = 4) | 1 (25%) | 3 (75%) | 0 (0%) |
| White women (n = 7) | 3 (43%) | 3 (43%) | 1 (14%) |
| DISEASE | | | |
| BREAST CANCER | | | |
| Black women (n = 8) | 8 (100%) | 0 (0%) | 0 (0%) |
| White women (n = 7) | 6 (86%) | 1 (14%) | 0 (0%) |
| LUNG CANCER | | | |
| Black men (n = 8) | 8 (100%) | 0 (0%) | 0 (0%) |
| Black women (n = 8) | 8 (100%) | 0 (0%) | 0 (0%) |
| White men (n = 8) | 8 (100%) | 0 (0%) | 0 (0%) |
| White women (n = 1) | 1 (100%) | 0 (0%) | 0 (0%) |
| PROSTATE CANCER | | | |
| Black men (n = 8) | 8 (100%) | 0 (0%) | 0 (0%) |
| White men (n = 8) | 8 (100%) | 0 (0%) | 0 (0%) |
| NIDDM | | | |
| Black men (n = 2) | 0 (0%) | 2 (100%) | 0 (0%) |
| Black women (n = 3) | 0 (0%) | 1 (33%) | 2 (67%) |
| White men (n = 4) | 0 (0%) | 2 (50%) | 2 (50%) |
| White women (n = 3) | 2 (67%) | 1 (33%) | 0 (0%) |
| ESRD due to NIDDM | | | |
| Black men (n = 6) | 6 (100%) | 0 (0%) | 0 (0%) |
| Black women (n = 8) | 8 (100%) | 0 (0%) | 0 (0%) |
| White men (n = 5) | 5 (100%) | 0 (0%) | 0 (0%) |
| White women (n = 4) | 4 (100%) | 0 (0%) | 0 (0%) |
| MYOCARDIAL INFARCTION | | | |
| White women (n = 7) | 7 (100%) | 0 (0%) | 0 (0%) |

Allele-Specific Odds Ratios

The susceptibility allele is indicated, as well as the odds ratio (OR). Haldane's correction was used if the denominator was zero. If the odds ratio (OR) was ≧1.5, the 95% confidence interval (C.I.) is also given. An odds ratio of 1.5 was chosen as the threshold of significance based on the recommendation of Austin et al. in Epidemiol. Rev., 16:65-76, (1994). “[E]pidemiology in general and case-control studies in particular are not well suited for detecting weak associations (odds ratios <1.5).” Id. at 66. Odds ratios of 1.5 or higher are high-lighted below.

TABLE 19
ALLELE-SPECIFIC ODDS RATIOS FOR GROUP I DISEASES

| | SUSCEPTIBILITY | | |
|---|---|---|---|
| DISEASE | ALLELE | OR | 95% C.I. |
| Breast Cancer | | | |
| Black women | G | 33 | 2.6-424 |
| White women | G | 5.2 | 1.3-21 |
| Lung Cancer | | | |
| Black men | G | 1.0 | |
| Black women | G | 33 | 2.6-424 |
| White men | G | 21 | 2.3-190 |
| White women | G | 2.9 | 0.3-28 |
| Prostate Cancer | | | |
| Black men | G | 1.0 | |
| White men | G | 21 | 2.3-190 |
| NIDDM | | | |
| Black men | A | 13 | 0.8-219 |
| Black women | A | 5.0 | 0.2-167 |
| White men | A | 5.0 | 0.6-43 |
| White women | G | 2.1 | 0.5-9.3 |
| ESRD due to NIDDM* | | | |
| Black men | G | 25 | 1.5-411 |
| Black women | G | 33 | 2.0-539 |
| White men | G | 63 | 4.8-820 |
| White women | G | 3.4 | 0.2-65 |
| Myocardial Infarction | | | |
| White women | G | 17 | 2.0-141 |

*Compared to group with NIDDM alone.

Genotype-Specific Odds Ratios

The susceptibility allele (S) is indicated, and the alternative allele at this locus is defined as the protective allele (P). Also presented is the odds ratio (OR) for the SS and SP genotypes. The odds ratio for the PP genotype is 1 by definition, since it is the reference group, and is not presented in the table below. For odds ratios ≧1.5, the asymptotic 95% confidence interval (C.I.) is also given, in parentheses. An odds ratio of 1.5 was chosen as the threshold of significance based on the recommendation of Austin et al., in Epidemiol. Rev., 16:65-76 (1994). “[E]pidemiology in general and case-control studies in particular are not well suited for detecting weak associations (odds ratios <1.5).” Id. at 66.

Odds ratios of 1.5 or higher are high-lighted below. Haldane's correction was used when the denominator was zero.

TABLE 20
GENOTYPE-SPECIFIC ODDS RATIOS FOR GROUP I DISEASES

| | SUSCEPTIBILITY | | |
|---|---|---|---|
| DISEASE | ALLELE | OR(SS) | OR(SP) |
| Breast Cancer | | | |
| Black women | G | 17 (0.6-524) | 0.3 |
| White women | G | 5.6 (0.5-64) | 1.3 |
| Lung Cancer | | | |
| Black women | G | 17 (0.6-524) | 0.3 |
| White men | G | 5.7 (0.3-118) | 0.1 |
| White women | G | 1.3 | 0.4 |
| Prostate Cancer | | | |
| White men | G | 5.7 (0.3-118) | 0.1 |
| NIDDM | | | |
| Black men | A | 7.0 (0.2-226) | 35 (1.7-703) |
| Black women | A | 5.0 (0.2-167) | 1.0 |
| White men | A | 15 (0.7-340) | 2.1 (0.2-27) |
| White women | G | 2.1 (0.2-27) | 1.3 |
| ESRD due | | | |
| to NIDDM* | | | |
| Black men | G | 13 (0.4-405) | 0.2 |
| Black women | G | 85 (4.5-1617) | 1.7 (0.1-38) |
| White men | G | 55 (2.8-1068) | 1.0 |
| White women | G | 1.8 (0.1-35) | 0.3 |
| Myocardial | | | |
| Infarction | | | |
| White women | G | 6.4 (0.6-73) | 0.4 |

*Compared to group with NIDDM alone.

PCR and sequencing were conducted as in Example 1. The primers were the same as in Example 1. The control samples agree with Hardy-Weinberg equilibrium, as follows:

A frequency of 1.0 for the G allele (“p”) and 0 for the A allele (“q”) among black male control individuals predicts genotype frequencies of 100% G/G, 0% G/A, and 0% A/A at Hardy-Weinberg equilibrium (p²+2pq+q²=1). The observed genotype frequencies were 100% G/G, 0% G/A, and 0% A/A, in perfect agreement with those predicted for Hardy-Weinberg equilibrium.

A frequency of 0.5 for the G allele (“p”) and 0.5 for the A allele (“q”) among black female control individuals predicts genotype frequencies of 25% G/G, 50% G/A, and 25% A/A at Hardy-Weinberg equilibrium (p²+2pq+q²=1). The observed genotype frequencies were 0% G/G, 100% G/A, and 0% A/A, in poor agreement with those predicted for Hardy-Weinberg equilibrium.

A frequency of 0.63 for the G allele (“p”) and 0.38 for the A allele (“q”) among white male control individuals predicts genotype frequencies of 40% G/G, 46% G/A, and 14% A/A at Hardy-Weinberg equilibrium (p²+2pq+q²=1). The observed genotype frequencies were 25% G/G, 75% G/A, and 0% A/A, in poor agreement with those predicted for Hardy-Weinberg equilibrium.

A frequency of 0.64 for the G allele (“p”) and 0.36 for the A allele (“q”) among white female control individuals predicts genotype frequencies of 41% G/G, 46% G/A, and 13% A/A at Hardy-Weinberg equilibrium (p²+2pq+q²=1). The observed genotype frequencies were 43% G/G, 43% G/A, and 14% A/A, in excellent agreement with those predicted for Hardy-Weinberg equilibrium.

Results

Using an allele-specific odds ratio of 1.5 or greater as a practical level of significance, the following observations can be made.

Among black women with breast cancer, the odds ratio for the G allele at this locus was a remarkable 33 (95% CI, 2.6-424). The odds ratio for the GA heterozygote was actually less than 1, and was 17 (95% CI, 0.6-524) for the GG homozygote. The genotype-specific odds ratios therefore suggest that the G allele behaves in a recessive fashion.

Among white women with breast cancer, the odds ratio for the G allele at this locus was 5.2 (95% CI, 1.3-21). The odds ratio for the GA heterozygote was 1.3, and was 5.6 (95% CI, 0.5-64) for the GG homozygote. The genotype-specific odds ratios therefore suggest that the G allele behaves in a recessive fashion.

For black women with lung cancer, the odds ratio for the G allele at this locus was a remarkable 33 (95% CI, 2.6-424). The odds ratio for the GA heterozygote was actually less than 1, and was 17 (95% CI, 0.6-524) for the GG homozygote. The genotype-specific odds ratios therefore suggest that the G allele behaves in a recessive fashion.

For white men with lung cancer, the odds ratio for the G allele at this locus was 21 (95% CI, 2.3-190). The odds ratio for the GA heterozygote was actually less than 1, and was 5.7-(95% CI, 0.3-118) for the GG homozygote. The genotype-specific odds ratios therefore suggest that the G allele behaves in a recessive fashion.

For white women with lung cancer, the odds ratio for the G allele at this locus was 2.9 (95% CI, 0.3-28). The genotype-specific odds ratios are unhelpful because neither the GA heterozygote nor GG homozygote has an odds ratio above 1.5. Thus, no inference can be drawn about whether the T allele behaves in a dominant, recessive, or codominant fashion.

For white men with prostate cancer, the odds ratio for the G allele at this locus was 21 (95% CI, 2.3-190). The odds ratio for the GA heterozygote was actually less than 1, and was 5.7 (95% CI, 0.3-118) for the GG homozygote. The genotype-specific odds ratios therefore suggest that the G allele behaves in a recessive fashion.

For black men with NIDDM, the odds ratio for the A allele was 13 (95% CI, 0.8-219). Inspection of the genotype-specific odds ratios suggests that the A allele was codominant, since the heterozygote had a much higher odds ratio (35, 95% CI, 1.7-703) than the AA homozygote (7.0, 95% CI, 0.2-226) or the reference GG genotype, the odds ratio of which equaled 1, by definition.

For black women with NIDDM, the odds ratio for the A allele at this locus was 5.0 (95% CI, 0.2-167). The odds ratio for the AG heterozygote was 1.0, and 5.0 (95% CI, 0.2-167) for the AA homozygote. The genotype-specific odds ratios therefore suggest that the A allele behaves in a recessive fashion.

For white men with NIDDM, the odds ratio for the A allele at this locus was 5.0 (95% CI, 0.6-43). The genotype-specific odds ratios suggest that the A allele behaves as a dominant susceptibility allele, since the heterozygote (with one allele copy) had an odds ratio of 2.1 (95% CI, 0.2-27). However, there is a pronounced (more than multiplicative) effect of gene dosage, since the homozygote with two copies of the A allele displayed a more than 7-fold larger odds ratio (15, 95% CI, 0.7-340).

For white women with NIDDM, the odds ratio for the G allele at this locus was 2.1 (95% CI, 0.5-9.3). The odds ratio for the GA heterozygote was 1.3, and was 2.1 (95% CI, 0.2-27) for the GG homozygote. The genotype-specific odds ratios therefore suggest that the G allele behaves in a recessive fashion.

For black men with ESRD due to NIDDM, the odds ratio for the G allele at this locus was 25 (95% CI, 1.5-411), compared to black men with NIDDM but no renal disease. The odds ratio for the GA heterozygote was actually less than 1, and was 13 (95% CI, 0.4-405) for the GG homozygote. The genotype-specific odds ratios therefore suggest that the G allele behaves in a recessive fashion.

For black women with ESRD due to NIDDM, the odds ratio for the G allele at this locus was 33 (95% CI, 2.0-539) relative to black women with NIDDM but normal kidney function. The genotype-specific odds ratios suggest that the G allele behaves as a dominant susceptibility allele, since the heterozygote (with one allele copy) had an odds ratio of 1.7 (95% CI, 0.1-38). However, there is a pronounced (more than multiplicative) effect of gene dosage, since the homozygote with two copies of the A allele displayed a 50-fold larger odds ratio (85, 95% CI, 4.5-1617).

For white men with ESRD due to NIDDM, the odds ratio for the G allele at this locus was 63 (95% CI, 4.8-820). The odds ratio for the GA heterozygote was 1.0, and was 55 (95% CI, 2.8-1068) for the GG homozygote. The genotype-specific odds ratios therefore suggest that the G allele behaves in a recessive fashion.

For white women with ESRD due to NIDDM, the odds ratio for the G allele at this locus was 3.4 (95% CI, 0.2-65). The odds ratio for the GA heterozygote was actually less than 1, and was 1.8 (95% CI, 0.1-35) for the GG homozygote. The genotype-specific odds ratios therefore suggest that the G allele behaves in a recessive fashion.

For white women with myocardial infarction, the odds ratio for the G allele at this locus was 17 (95% CI, 2.0-141). The odds ratio for the GA heterozygote was actually less than 1, and was 6.4 (95% CI, 0.6-73) for the GG homozygote. The genotype-specific odds ratios therefore suggest that the G allele behaves in a recessive fashion.

Analysis

According to commercially available software [GENOMATIX MatInspector Professional; URL: http://genomatix.gsf.de/cgi-bin/matinspector/matinspector.pl; Quandt et al., Nucleic Acids Res. 23: 4878-4884 (1995)], the G2701→A SNP is predicted to have the following potential effects on transcription of the ecNOS gene:
a. Disruption of the binding site for AP4_Q5 (activating protein 4), whose binding site consists of the sequence 5′-NNCAGCTGNN-3′(SEQ ID NO: 12), beginning at position 2697 on the (+) strand. The G2701→A SNP replaces the indicated G in the core binding site with an A. AP4_Q6 sites occur relatively rarely: 0.50 sites per 1,000 base pairs of random genomic sequence in vertebrates.
b. Disruption of the binding site for NFE2_{—}01 (NF-E2 p45), which consists of the complementary sequence to 5′-RTGASTCAGCA-3′(SEQ ID NO: 13), ending at position 2693 on the (−) strand. This SNP replaces the indicated G in the core binding site with an A. NFE2_{—}01 sites occur even less often than AP4_Q5 sites: 0.12 sites per 1,000 base pairs of random genomic sequence in vertebrates.

Both AP4 and NF-E2 are positive transcriptional regulators which activate expression of a gene. The G allele is expected to result in more efficient binding by AP4_Q6 and/or NFE2_{—}01 than the A allele, with the result that more ecNOS is expressed by patients carrying the G allele than the A allele. Tissue NO levels are therefore expected to be higher for G allele carrying individuals than for those bearing the A allele.

The universal presence of the G allele among white and black patients of both genders with ESRD due to NIDDM, for example, suggests that increased NO may be associated with the progression of diabetic nephropathy to end-stage renal disease.

### Example 5

### G to A Transition at Position 2843 of Human ecNOS Promoter

TABLE 21
ALLELE FREQUENCIES FOR GROUP I DISEASES

| | G | A |
|---|---|---|
| CONTROL | | |
| Black men (n = 4 chromosomes) | 4 (100%) | 0 (0%) |
| White men (n = 2 chromosomes) | 2 (100%) | 0 (0%) |
| White women (n = 8 chromosomes) | 7 (88%) | 1 (13%) |
| DISEASE | | |
| LUNG CANCER | | |
| Black men (n = 16 chromosomes) | 16 (100%) | 0 (0%) |
| Black women (n = 16 chromosomes) | 16 (100%) | 0 (0%) |
| White men (n = 16 chromosomes) | 16 (100%) | 0 (0%) |
| White women (n = 2 chromosomes) | 2 (100%) | 0 (0%) |
| PROSTATE CANCER | | |
| Black men (n = 14 chromosomes) | 14 (100%) | 0 (0%) |
| White men (n = 16 chromosomes) | 16 (100%) | 0 (0%) |
| ESRD due to NIDDM | | |
| Black men (n = 12 chromosomes) | 12 (100%) | 0 (0%) |
| Black women (n = 16 chromosomes) | 16 (100%) | 0 (0%) |
| White men (n = 10 chromosomes) | 10 (100%) | 0 (0%) |
| White women (n = 8 chromosomes) | 8 (100%) | 0 (0%) |
| NIDDM | | |
| Black men (n = 4 chromosomes) | 4 (100%) | 0 (0%) |
| Black women (n = 4 chromosomes) | 4 (100%) | 0 (0%) |
| White men (n = 8 chromosomes) | 8 (100%) | 0 (0%) |
| White women (n = 6 chromosomes) | 6 (100%) | 0 (0%) |
| MYOCARDIAL INFARCTION | | |
| White women (n = 14 chromosomes) | 14 (100%) | 0 (0%) |

TABLE 22
GENOTYPE FREQUENCIES FOR GROUP I DISEASES

| | G/G | G/A | A/A |
|---|---|---|---|
| CONTROLS | | | |
| Black men (n = 2) | 2 (100%) | 0 (0%) | 0 (0%) |
| White men (n = 1) | 1 (100%) | 0 (0%) | 0 (0%) |
| White women (n = 4) | 3 (75%) | 1 (25%) | 0 (0%) |
| DISEASE | | | |
| LUNG CANCER | | | |
| Black men (n = 8) | 8 (100%) | 0 (0%) | 0 (0%) |
| Black women (n = 8) | 8 (100%) | 0 (0%) | 0 (0%) |
| White men (n = 8) | 8 (100%) | 0 (0%) | 0 (0%) |
| White women (n = 1) | 1 (100%) | 0 (0%) | 0 (0%) |
| PROSTATE CANCER | | | |
| Black men (n = 7) | 7 (100%) | 0 (0%) | 0 (0%) |
| White men (n = 8) | 8 (100%) | 0 (0%) | 0 (0%) |
| ESRD due to NIDDM | | | |
| Black men (n = 6) | 6 (100%) | 0 (0%) | 0 (0%) |
| Black women (n = 8) | 8 (100%) | 0 (0%) | 0 (0%) |
| White men (n = 5) | 5 (100%) | 0 (0%) | 0 (0%) |
| White women (n = 4) | 4 (100%) | 0 (0%) | 0 (0%) |
| NIDDM | | | |
| Black men (n = 2) | 2 (100%) | 0 (0%) | 0 (0%) |
| Black women (n = 2) | 2 (100%) | 0 (0%) | 0 (0%) |
| White men (n = 4) | 4 (100%) | 0 (0%) | 0 (0%) |
| White women (n = 3) | 3 (100%) | 0 (0%) | 0 (0%) |
| MYOCARDIAL INFARCTION | | | |
| White women (n = 7) | 7 (100%) | 0 (0%) | 0 (0%) |

Allele-Specific Odds Ratios

The susceptibility allele is indicated, as well as the odds ratio (OR). Haldane's correction was used if the denominator was zero. If the odds ratio (OR) was ≧1.5, the 95% confidence interval (C.I.) is also given. An odds ratio of 1.5 was chosen as the threshold of significance based on the recommendation of Austin et al. (Epidemiol Rev. 16:65-76, 1994). “. . .[E]pidemiology in general and case-control studies in particular are not well suited for detecting weak associations (odds ratios <1.5)[p. 66].” Odds ratios of 1.5 or higher are high-lighted below.

A black female control group was not available. We therefore used the black female lung cancer group as a putative control group, since the frequency of the G allele appears to be 100% for black male controls as well as black men and women in most disease categories.

TABLE 23
ALLELE-SPECIFIC ODDS RATIOS FOR GROUP I DISEASES

| | SUSCEPTIBILITY | | |
|---|---|---|---|
| DISEASE | ALLELE | OR | 95% C.I. |
| Lung Cancer | | | |
| Black men | G | 1.0 | |
| Black women | G | 1 (ref. Group) | |
| White men | G | 1.0 | |
| White women | G | 1.0 | |
| Prostate Cancer | | | |
| Black men | G | 1.0 | |
| White men | G | 1.0 | |
| NIDDM | | | |
| Black men | G | 1.0 | |
| Black women | G | 1.0 | |
| White men | G | 1.0 | |
| White women | G | 2.6 | 0.2-28 |
| ESRD due | | | |
| to NIDDM* | | | |
| Black men | G | 1.0 | |
| Black women | G | 1.0 | |
| White men | G | 1.0 | |
| White women | G | 1.0 | |
| Myocardial | | | |
| Infarction | | | |
| White women | G | 5.8 | 0.6-61 |

*Compared to group with NIDDM alone.

Genotype-Specific Odds Ratios

The susceptibility allele (S) is indicated, and the alternative allele at this locus is defined as the protective allele (P). Also presented is the odds ratio (OR) for the SS and SP genotypes. The odds ratio for the PP genotype is 1 by definition, since it is the reference group, and is not presented in the table below. For odds ratios ≧1.5, the asymptotic 95% confidence interval (C.I.) is also given, in parentheses. An odds ratio of 1.5 was chosen as the threshold of significance based on the recommendation of Austin et al., in Epidemiol. Rev., 16:65-76 (1994). “[E]pidemiology in general and case-control studies in particular are not well suited for detecting weak associations (odds ratios <1.5).” Id. at 66.

Odds ratios of 1.5 or higher are high-lighted below. Haldane's correction was used when the denominator was zero.

TABLE 24
GENOTYPE-SPECIFIC ODDS RATIO FOR GROUP I DISEASES

| | SUSCEPTIBILITY | | |
|---|---|---|---|
| DISEASE | ALLELE | OR(SS) | OR(SP) |
| NIDDM | | | |
| White women | G | 1.0 | 0.3 |
| Myocardial Infarction | | | |
| White women | G | 2.1 (0.1-40) | 0.3 |

PCR and sequencing were conducted as in Example 1. The primers were the same as in Example 1. The control samples agree with Hardy-Weinberg equilibrium, as follows:

A frequency of 1.0 for the G allele (“p”) and 0 for the A allele (“q”) among black male control individuals predicts genotype frequencies of 100% G/G, 0% G/A, and 0% A/A at Hardy-Weinberg equilibrium (p²+2pq+q²=1). The observed genotype frequencies were 100% G/G, 0% G/A, and 0% A/A, in perfect agreement with those predicted for Hardy-Weinberg equilibrium.

A frequency of 1.0 for the G allele (“p”) and 0 for the A allele (“q”) among white male control individuals predicts genotype frequencies of 100% G/G, 0% G/A, and 0% A/A at Hardy-Weinberg equilibrium (p²+2pq+q²=1). The observed genotype frequencies were 100% G/G, 0% G/A, and 0% A/A, in perfect agreement with those predicted for Hardy-Weinberg equilibrium.

A frequency of 0.88 for the G allele (“p”) and 0.13 for the A allele (“q”) among white female control individuals predicts genotype frequencies of 77% G/G, 21% G/A, and 2% A/A at Hardy-Weinberg equilibrium (p²+2pq+q²=1). The observed genotype frequencies were 75% G/G, 25% G/A, and 0% A/A, in good agreement with those predicted for Hardy-Weinberg equilibrium.

Results

For white women with NIDDM, the odds ratio for the G allele at this locus was 2.6 (95% CI, 0.2-28). The genotype-specific odds ratios were unhelpful because neither the GA heterozygote nor GG homozygote has an odds ratio above 1.5. Thus, no inference can be drawn about whether the T allele behaves in a dominant, recessive, or codominant fashion.

For white women with myocardial infarction, the odds ratio for the G allele at this locus was 5.8 (95% CI, 0.6-61). The odds ratio for the GA heterozygote was actually less than 1, and 2.1 (95% CI, 0.1-40) for the GG homozygote. The genotype-specific odds ratios therefore suggest that the G allele behaves in a recessive fashion.

Analysis

According to commercially available software [GENOMATIX Matinspector Professional; URL: http://genomatix.gsf.de/cgi-bin/matinspector/matinspector.pl; Quandt et al., Nucleic Acids Res. 23: 4878-4884 (1995)], this SNP is predicted to disrupt a potential binding site for NFY_Q6 (nuclear factor Y [Y-box binding factor]). Its binding site consists of the sequence complementary to 5′-NYSATTGGYYA-3′ (SEQ ID NO: 14), ending at position 2837 on the (−) strand. The G2843→A SNP replaces the indicated G in the core binding site with an A. NFY_Q6 sites occur relatively rarely: 0.70 sites per 1,000 base pairs of random genomic sequence in vertebrates.

NFY, also called CP1 (Stewart et al., Gene., 173(2):155-161, 1996) is a positive transcriptional regulator which activates gene expression. NFY has not yet been implicated in regulation of ecNOS gene expression. The G allele is expected to result in more efficient binding by NFY than the A allele, with the result that more ecNOS is expressed by patients carrying the G allele than the A allele. Tissue NO levels are therefore expected to be higher for G allele carrying individuals than for those bearing the A allele. Higher tissue NO levels therefore appear to predispose white women specifically to NIDDM, lung cancer, and myocardial infarction.

TABLE 25

| Gene | Region | Location | Wild Type | Variant | SEQ ID |
|---|---|---|---|---|---|
| ecNOS | Promoter | 2548 | G | A | 1 |
| | | 2684 | C | T | 1 |
| | | 2701 | G | A | 1 |
| | | 2843 | G | A | 1 |

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

1. A method for diagnosing a genetic susceptibility for a disease, condition, or disorder in a subject comprising:
obtaining a biological sample containing nucleic acid from said subject; and analyzing said nucleic acid to detect the presence or absence of a single nucleotide polymorphism in the ecNOS gene, wherein said single nucleotide polymorphism is associated with a genetic predisposition for a disease, condition or disorder selected from the group consisting of breast cancer, lung cancer, prostate cancer, non-insulin dependent diabetes, end stage renal disease due to non-insulin dependent diabetes, hypertension, end stage renal disease due to hypertension, myocardial infarction, colon cancer, hypertension, atherosclerotic peripheral vascular disease due to hypertension, cerebrovascular accident due to hypertension, cataracts due to hypertension, cardiomyopathy with hypertension, myocardial infarction due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder.

2. The method of claim 1, wherein the gene ecNOS comprises SEQ ID NO: 1.

3. The method of claim 1, wherein said nucleic acid is DNA, RNA, cDNA or MRNA.

4. The method of claim 2, wherein said single nucleotide polymorphism is located at position 2548, 2684, 2701, or 2843 of SEQ ID NO: 1.

5. The method of claim 4, wherein said single nucleotide polymorphism is selected from the group consisting of G2548→A, C2684→T, G2701→A, and G2843→A and its complements namely C2548→T, G2684→A, C2701→T, and C2843→T.

6. The method of claim 1, wherein said analysis is accomplished by sequencing, mini sequencing, hybridization, restriction fragment analysis, oligonucleotide ligation assay or allele specific PCR.

7. An isolated polynucleotide comprising at least 10 contiguous nucleotides of SEQ ID NO: 1, or the complement thereof, and containing at least one single nucleotide polymorphism at position 2548, 2684, 2701, or 2843 of SEQ ID NO: 1 wherein said at least one single nucleotide polymorphism is associated with a disease, condition or disorder selected from the group consisting of breast cancer, lung cancer, prostate cancer, non-insulin dependent diabetes, end stage renal disease due to non-insulin dependent diabetes, hypertension, end stage renal disease due to hypertension, myocardial infarction, colon cancer, hypertension, atherosclerotic peripheral vascular disease due to hypertension, cerebrovascular accident due to hypertension, cataracts due to hypertension, cardiomyopathy with hypertension, myocardial infarction due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder.

8. The isolated polynucleotide of claim 7, wherein at least one single nucleotide polymorphism is selected from the group consisting of G2548→A, C2684→T, G2701→A, and G2843→A and its complements namely C2548→T, G2684→A, C2701→T, and C2843→T.

9. The isolated polynucleotide of claim 7, wherein said at least one single nucleotide polymorphism is located at the 3′ end of said nucleic acid sequence.

10. The isolated polynucleotide of claim 7, further comprising a detectable label.

11. The isolated nucleic acid sequence of claim 10, wherein said detectable label is selected from the group consisting of radionuclides, fluorophores or fluorochromes, peptides, enzymes, antigens, antibodies, vitamins or steroids.

12. A kit comprising at least one isolated polynucleotide of at least 10 contiguous nucleotides of SEQ ID NO: 1 or the complement thereof, and containing at least one single nucleotide polymorphism associated with a disease, condition, or disorder selected from the group consisting of breast cancer, lung cancer, prostate cancer, non-insulin dependent diabetes, end stage renal disease due to non-insulin dependent diabetes, hypertension, end stage renal disease due to hypertension, myocardial infarction, colon cancer, hypertension, atherosclerotic peripheral vascular disease due to hypertension, cerebrovascular accident due to hypertension, cataracts due to hypertension, cardiomyopathy with hypertension, myocardial infarction due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder; and instructions for using said polynucleotide for detecting the presence or absence of said at least one single nucleotide polymorphism in said nucleic acid.

13. The kit of claim 12 wherein said at least one single nucleotide polymorphism is located at position 2548, 2684, 2701, or 2843 of SEQ ID NO: 1.

14. The kit of claim 13 wherein said at least one single nucleotide polymorphism is selected from the group consisting of G2548→A, C2684→T, G2701→A, and G2843→A and its complements namely C2548→T, G2684→A, C2701→T, and C2843→T.

15. The kit of claim 12, wherein said single nucleotide polymorphism is located at the 3′ end of said polynucleotide.

16. The kit of claim 12, wherein said polynucleotide further comprises at least one detectable label.

17. The kit of claim 16, wherein said label is chosen from the group consisting of radionuclides, fluorophores or fluorochromes, peptides enzymes, antigens, antibodies, vitamins or steroids.

18. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease, condition, or disorder selected from the group consisting of breast cancer, lung cancer, prostate cancer, non-insulin dependent diabetes, end stage renal disease due to non-insulin dependent diabetes, hypertension, end stage renal disease due to hypertension, myocardial infarction, colon cancer, hypertension, atherosclerotic peripheral vascular disease due to hypertension, cerebrovascular accident due to hypertension, cataracts due to hypertension, cardiomyopathy with hypertension, myocardial infarction due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder; and instructions for using said polynucleotide for detecting the presence or absence of said single nucleotide polymorphism in a biological sample containing nucleic acid.

19. The kit of claim 18, wherein said single nucleotide polymorphism site is located at position 2548, 2684, 2701, or 2843 of SEQ ID NO: 1.

20. The kit of claim 19, wherein said at least one polynucleotide further comprises a detectable label.

21. The kit of claim 20, wherein said detectable label is chosen from the group consisting of radionuclides, fluorophores or fluorochromes, peptides, enzymes, antigens, antibodies, vitamins or steroids.

22. A method for treatment or prophylaxis in a subject comprising:
obtaining a sample of biological material containing nucleic acid from a subject;
analyzing said nucleic acid to detect the presence or absence of at least one single nucleotide polymorphism in SEQ ID NO: 1 or the complement thereof associated with a disease, condition, or disorder selected from the group consisting of breast cancer, lung cancer, prostate cancer, non-insulin dependent diabetes, end stage renal disease due to non-insulin dependent diabetes, hypertension, end stage renal disease due to hypertension, myocardial infarction, colon cancer, hypertension, atherosclerotic peripheral vascular disease due to hypertension, cerebrovascular accident due to hypertension, cataracts due to hypertension, cardiomyopathy with hypertension, myocardial infarction due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, or seizure disorder; and
treating said subject for said disease, condition or disorder.

23. The method of claim 22 wherein said nucleic acid is selected from the group consisting of DNA, CDNA, RNA and MRNA.

24. The method of claim 22, wherein said at least one single nucleotide polymorphism is located at position 2548, 2684, 2701, or 2843 of SEQ ID NO: 1.

25. The method of claim 22 wherein said at least one single nucleotide polymorphism is selected from the group consisting of G2548→A, C2684→T, G2701→A, and G2843→A and its complements namely C2548→T, G2684→A, C2701→T, and C2843→T.

26. The method of claim 22 wherein said treatment counteracts the effect of said at least one single nucleotide polymorphism detected.


SEQUENCE LISTING (14 sequences)

SEQ ID NO: 1 - DNA, 3585 bp, Homo sapiens
gggcccagagaaagagctgtccccggggccttggggacagggtgacagccacccagagatcatggagaaggggacgtaaggaagacctcacagaggagtcatcctgcgactgtgttggttgggtccttcaggaagcagagtcccaggagttggaagcataagaggaatactgcgggcaatgcctgagaaagataacagggaccgggagcaggagtgagttgggcaggggaaggatcaggcccacaatgccaggctcacacctgcagaggagggaagaagaagaagggcctcacatcagcccagcgggggatgttacgcccacagacgccccggggctcagttactgtctaagtgttagaaataaattttcggtgccacaaaagaaatagcactcagattaaatgttcccagcaaggcaattttacttctatagaagggtgcatctcacagatggagcaatggcaagagcacacctgaacaagggaagggaaggggtttttatccctaaggcaggtagcccctacagctgtgttgttcccctattggctagggttggaccacaccgtctgagctaattgttactggctattttaaagagagcaggggtaagagccggattggcagggtaagtagtttggcaggaaggacggtcacagaacaggtgactcaggatgactcaggtcagagcaggtgaccagtggtgactcagttcggagcaggtgatagaagctaggagggggttgtttactgaaactaggggcaaggagacgaagagaacatgaaagttaaactttaagatgaagaacaaagctgaacatactgatgcattggatctttggagaggatctcagaactcattgtacttaatttacaggctaaaaccttagaagaggaatttattatatcctacacaagactccagggaagcacatggccttggactgaaggctggcatctggaagctgtcagccaccagcaccttctgcagcaggtacctgctctctaagagggaggcctgggtggtgcacctccagagctgcccaggctgggcctcaaggaagaaaaagattttcatttgtcagaggcggaagggagaggtggagggaacagcacagcagcggcccaggggcagggaagcacaggaccattagggagacacgagaaagcccatttgtctagaacagaggattcaagcagtgcaccaaggaaaatgagggccaggccaatgtgctggagtggctttgttcttggctgagggttttgggtagtgccaaagcgtaaggtaagccctgctttccagaagaatctagcagagtgtggagcccagatgggactggaaggcctgggaggggtcaggtggccacagggacgggccacagccagtggtgcaggcaagaagacaatggccatccatggtggctcacacctggaatcccagcccattgggaggtcgaggcaggtggatcacctgaggtcaggagttcgagaccagcctggtcaacatggtgaaaccctgtctctaataaaattataaaaattagccgggcgtggtggtgggtacctgtaatctcagctactcaggaggctgggtcaggagaatcgcttgaacccaggaggcggaggttacagtgagctgagatagcaccattgcattccagcctggacaacaaaagcgagactctgtctcaaaaaaaaaaaaaaattagccaggcgtggtggtgggtgcctgtcgtcctcgggaggctgaggcatgagaatcactccgggaggcagaggttgcaatgaaccaagatcacaccactgcactccagcctgggtgacagagcaagactctgtctaaaaaaaaaaaaaagacagaaggatgtcagcatctgatgctgcctgtcaccttgaccctgaggatgccagtcacagctccattaactgggacctaggaaaatgagtcatccttggtcatgcacatttcaaatggtggcttaatatggaagccacacttgggatctgttgtctcctccagcatggtagaagatgcctgaaaagtaggggctggatcccatcccctgcctcactgggaaggcgaggtggtggggtggggtggggcctcaggcttggggtcatgggacaaagcccaggctgaatgccgcccttccatctccctcctcctgagacaggggcagcagggcacactagtgtccaggagcagcttatgaggccccttcaccctccgatcctccaaaactggcagaccccaccttcttcggtgtgaccccagagctctgagcacagcccgttccttccgcctgccggccccccacccaggcccaccccaaccttatcctccactgcttttcagaggagtctggccaacacaaatcctcttgtttgtttgtctgtctgtctgctgctcctagtctctgcctctcccagtctctcagcttccgtttctttcttaaactttctctcagtctctgaggtctcgaaatcacgaggcttcgacccctgtggaccagatgcccagctagtggcctttctccagcccctcagatggcacagaactacaaaccccagcatgcactctggcctgaagtgcctggagagtgctggtgtaccccacctgcattctgggaactgtagtttccctagtcccccatgctcccaccagggcatcaagctcttccctggccggctgaccctgcctcagccctagtctctctgctgacctgcggccccgggaagcgtgcgtcactgaatgacagggtgggggtggaggcactggaaggcagcttcctgctcttttgtgtcccccacttgagtcatgggggtgtgggggttccaggaaattggggctgggaggggaagggataccctaatgtcagactcaaggacaaaaagtcactacatccttgctgggcctctatccccaagaacccaaaaggactcaagggtggggatccaggagttcttgtatgtatggggggaggtgaaggagagaacctgcatgaccctagaggtccctgtggtcactgagagtgtgggctgccatcccctgctacagaaacggtgctcaccttctgcccaaccctccagggaaaggcacacaggggtgaggccgaaccttccgtctggtgccacatcacagaaggacctttatgaccccctggtggctctaccctgccactccccaatgccccagcccccatgctgcagccccagggctctgctggacacctgggctcccacttatcagcctcagtcctcacagcggaacccaggcgtccggccccccacccttcaggccagcgggcgtggagctgaggctttagagcctcccagccgggcttgttcctgtcccattgtgtatgggataggggcggggcgagggccagcactggagagccccctcccactgccccctcctctcggtcccctccctcttcctaaggaaaaggccagggctctgctggagcaggcagcagagtggacgcacagtaacatgggcaacttgaagagcgtggcccaggagcctgggccaccctgcggcctggggctggggctgggccttgggctgtgcggcaagcagggccc

SEQ ID NO: 2 - DNA, 21 bp, Artificial sequence
  Primer
gagtctggccaacacaaatcc

SEQ ID NO: 3 - DNA, 22 bp, Artificial sequence
  Primer
ctctagggtcatgcaggttctc

SEQ ID NO: 4 - DNA, 18 bp, Homo sapiens
  variation (6)..(6): Polymorphism results in G being replaced by A at this position
agatggcacagaactaca

SEQ ID NO: 5 - DNA, 10 bp, Homo sapiens
  variation (2)..(2): Polymorphism results in C being replaced by T on (-) strand and A on the (+) strand at this position
gccatctgag

SEQ ID NO: 6 - DNA, 12 bp, Homo sapiens
  variation (10)..(10): Polymorphism results in G being replaced by A at this position
cctcagatggca

SEQ ID NO: 7 - DNA, 16 bp, Homo sapiens
  variation (12)..(12): Polymorphism results in G being replaced by A at this position
cccctcagatggcaca

SEQ ID NO: 8 - DNA, 18 bp, Homo sapiens
  variation (8)..(8): Polymorphism results in C being replaced by T at this position
ccctggccggctgaccct

SEQ ID NO: 9 - DNA, 13 bp, Homo sapiens
  variation (8)..(8): Polymorphism results in G being replaced by A on the (-) strand at this position
gtcagccggccag

SEQ ID NO: 10 - DNA, 10 bp, Homo sapiens
  variation (9)..(9): Polymorphism results in G being replaced by A on the (-) strand at this position
gtcagccggc

SEQ ID NO: 11 - DNA, 19 bp, Homo sapiens
  variation (3)..(3): Polymorphism results in C being replaced by T at this position
gccggctgaccctgcctca

SEQ ID NO: 12 - DNA, 10 bp, Homo sapiens
  variation (5)..(5): SNP replaces the G in the core binding site with an A at this position
nncagctgnn

SEQ ID NO: 13 - DNA, 11 bp, Homo sapiens
  variation (9)..(9): SNP replaces the G in the core binding site with an A at this position
rtgastcagca

SEQ ID NO: 14 - DNA, 11 bp, Homo sapiens
  variation (8)..(8): SNP replaces G in the core binding site with an A at this position
nysattggyya